SEQUENCE LISTING (55 sequences)

SEQ ID NO: 1 - DNA, 20 bp, Artificial Sequence
  Description of Artificial Sequence Synthetic primer
cagggttaatagggcctggt

SEQ ID NO: 2 - DNA, 23 bp, Artificial Sequence
  Description of Artificial Sequence Synthetic primer
gaaaaatttctagagggggtctg

SEQ ID NO: 3 - DNA, 20 bp, Artificial Sequence
  Description of Artificial Sequence Synthetic primer
ctctgtctctcctgcccaat

SEQ ID NO: 4 - DNA, 21 bp, Artificial Sequence
  Description of Artificial Sequence Synthetic primer
gctaattcaaatgcttggttg

SEQ ID NO: 5 - DNA, 20 bp, Artificial Sequence
  Description of Artificial Sequence Synthetic primer
aggtggttggagatcacctg

SEQ ID NO: 6 - DNA, 20 bp, Artificial Sequence
  Description of Artificial Sequence Synthetic primer
ctttcgcaagaaccacttcc

SEQ ID NO: 7 - DNA, 25 bp, Artificial Sequence
  Description of Artificial Sequence Synthetic primer
tgggtaatttagaggaacaaagtca

SEQ ID NO: 8 - DNA, 24 bp, Artificial Sequence
  Description of Artificial Sequence Synthetic primer
acacatcactcatagaaaaccaga

SEQ ID NO: 9 - DNA, 22 bp, Artificial Sequence
  Description of Artificial Sequence Synthetic primer
catcgtcttcttgctgttagga

SEQ ID NO: 10 - DNA, 22 bp, Artificial Sequence
  Description of Artificial Sequence Synthetic primer
ggtcttggttcaggtagggaga

SEQ ID NO: 11 - DNA, 21 bp, Artificial Sequence
  Description of Artificial Sequence Synthetic primer
ctgcaatctgcacatgaaaag

SEQ ID NO: 12 - DNA, 21 bp, Artificial Sequence
  Description of Artificial Sequence Synthetic primer
tgcatgaaagcacaaggtaaa

SEQ ID NO: 13 - DNA, 21 bp, Artificial Sequence
  Description of Artificial Sequence Synthetic primer
gtcagatggagttacgcaggt

SEQ ID NO: 14 - DNA, 25 bp, Artificial Sequence
  Description of Artificial Sequence Synthetic primer
aatccattttcactaaaagtgtgtg

SEQ ID NO: 15 - DNA, 20 bp, Artificial Sequence
  Description of Artificial Sequence Synthetic primer
acgcctctttggtctccttg

SEQ ID NO: 16 - DNA, 20 bp, Artificial Sequence
  Description of Artificial Sequence Synthetic primer
tccatcccaagtccctttag

SEQ ID NO: 17 - DNA, 18 bp, Artificial Sequence
  Description of Artificial Sequence Synthetic primer
ctgcagagccctgtagcc

SEQ ID NO: 18 - DNA, 20 bp, Artificial Sequence
  Description of Artificial Sequence Synthetic primer
tgacgatatgggcttgtgag

SEQ ID NO: 19 - DNA, 20 bp, Artificial Sequence
  Description of Artificial Sequence Synthetic primer
ttaagaagggggagttgtgg

SEQ ID NO: 20 - DNA, 20 bp, Artificial Sequence
  Description of Artificial Sequence Synthetic primer
ggtgctatgggaaatgcaaa

SEQ ID NO: 21 - DNA, 20 bp, Artificial Sequence
  Description of Artificial Sequence Synthetic primer
cgactccacagcatctctga

SEQ ID NO: 22 - DNA, 24 bp, Artificial Sequence
  Description of Artificial Sequence Synthetic primer
tctcctcctcttatcagctctctc

SEQ ID NO: 23 - DNA, 21 bp, Artificial Sequence
  Description of Artificial Sequence Synthetic primer
gccacgtgatcattctctagc

SEQ ID NO: 24 - DNA, 22 bp, Artificial Sequence
  Description of Artificial Sequence Synthetic primer
ttcctttgggaaagatcaaaaa

SEQ ID NO: 25 - DNA, 20 bp, Artificial Sequence
  Description of Artificial Sequence Synthetic primer
ccagaacaatgatgggcttt

SEQ ID NO: 26 - DNA, 20 bp, Artificial Sequence
  Description of Artificial Sequence Synthetic primer
cactcgctcagagcaggtta

SEQ ID NO: 27 - DNA, 21 bp, Artificial Sequence
  Description of Artificial Sequence Synthetic primer
gggaaatagcaaatggacaca

SEQ ID NO: 28 - DNA, 22 bp, Artificial Sequence
  Description of Artificial Sequence Synthetic primer
attggtaaagccagtttcaagc

SEQ ID NO: 29 - DNA, 18 bp, Artificial Sequence
  Description of Artificial Sequence Synthetic primer
ggagggaggaggagttgg

SEQ ID NO: 30 - DNA, 20 bp, Artificial Sequence
  Description of Artificial Sequence Synthetic primer
cgtcttctcccagacacacc

SEQ ID NO: 31 - DNA, 23 bp, Artificial Sequence
  Description of Artificial Sequence Synthetic primer
gcacagagtaattcggtacttga

SEQ ID NO: 32 - DNA, 20 bp, Artificial Sequence
  Description of Artificial Sequence Synthetic primer
ttgccattccctaagcattt

SEQ ID NO: 33 - DNA, 22 bp, Artificial Sequence
  Description of Artificial Sequence Synthetic primer
aatcatctgacattttgcctct

SEQ ID NO: 34 - DNA, 20 bp, Artificial Sequence
  Description of Artificial Sequence Synthetic primer
agctgttgaagcaatcatgc

SEQ ID NO: 35 - DNA, 21 bp, Artificial Sequence
  Description of Artificial Sequence Synthetic primer
ccaaaaacagctgtcagaacc

SEQ ID NO: 36 - DNA, 22 bp, Artificial Sequence
  Description of Artificial Sequence Synthetic primer
ggctgccattaaacaagtaagc

SEQ ID NO: 37 - DNA, 18 bp, Artificial Sequence
  Description of Artificial Sequence Synthetic primer
cctggtgcccactcattg

SEQ ID NO: 38 - DNA, 19 bp, Artificial Sequence
  Description of Artificial Sequence Synthetic primer
cgacactgctggacaggaa

SEQ ID NO: 39 - DNA, 20 bp, Artificial Sequence
  Description of Artificial Sequence Synthetic primer
cagtgttgtgggatttgcac

SEQ ID NO: 40 - DNA, 27 bp, Artificial Sequence
  Description of Artificial Sequence Synthetic primer
gggtaactgataatttttctcactttt

SEQ ID NO: 41 - DNA, 22 bp, Artificial Sequence
  Description of Artificial Sequence Synthetic primer
cctcccagagtacttccaactc

SEQ ID NO: 42 - DNA, 20 bp, Artificial Sequence
  Description of Artificial Sequence Synthetic primer
accacgaagagactggctgt

SEQ ID NO: 43 - DNA, 22 bp, Artificial Sequence
  Description of Artificial Sequence Synthetic primer
caaatgtggctattctgatcca

SEQ ID NO: 44 - DNA, 23 bp, Artificial Sequence
  Description of Artificial Sequence Synthetic primer
ggcaaaaatgtaaaggagtcaaa

SEQ ID NO: 45 - DNA, 20 bp, Artificial Sequence
  Description of Artificial Sequence Synthetic primer
ggttgccatgtcattggttt

SEQ ID NO: 46 - DNA, 20 bp, Artificial Sequence
  Description of Artificial Sequence Synthetic primer
agggcacagagcaaaatgat

SEQ ID NO: 47 - DNA, 22 bp, Artificial Sequence
  Description of Artificial Sequence Synthetic primer
gaaaaggctgaaaacctcttga

SEQ ID NO: 48 - DNA, 20 bp, Artificial Sequence
  Description of Artificial Sequence Synthetic primer
tttttgatctgggctcactt

SEQ ID NO: 49 - DNA, 21 bp, Artificial Sequence
  Description of Artificial Sequence Synthetic primer
gataatactcaccgccagcag

SEQ ID NO: 50 - DNA, 20 bp, Artificial Sequence
  Description of Artificial Sequence Synthetic primer
ggatgttgacaaggcagtga

SEQ ID NO: 51 - DNA, 21 bp, Artificial Sequence
  Description of Artificial Sequence Synthetic primer
tccatgaagcacaaaacacaa

SEQ ID NO: 52 - DNA, 20 bp, Artificial Sequence
  Description of Artificial Sequence Synthetic primer
gtttggggcactccttcaac

SEQ ID NO: 53 - DNA, 52 bp, Homo sapiens
ttccatagcctaatccctgggcattgytttccactgaggttgggggttgggg

SEQ ID NO: 54 - DNA, 52 bp, Homo sapiens
ggtaataatgttgtctttttttttccyggcaaactttctgctttcggtccag

SEQ ID NO: 55 - DNA, 52 bp, Homo sapiens
ggcagccctggagcgtgggttctctgygaaatattctgcaaaatttgaaacg

(19) United States
(12) Patent Application Publication
(10) Pub. No.: US 2013/0023430 A1
(43) Pub. Date: Jan. 24, 2013

(54) CANCER STEM CELL GENE VARIANTS ARE ASSOCIATED WITH TUMOR RECURRENCE

(71) Applicant: Heinz-Josef Lenz, Altadena, CA (US)

(72) Inventor: Heinz-Josef Lenz, Altadena, CA (US)

(73) Assignee: University of Southern California

(21) Appl. No.: 13/470,174

(22) Filed: May 11, 2012

Related U.S. Application Data
(60) Provisional application No. 61/486,168, filed on May 13, 2011.

(51) Int. Cl. C12Q 1/68; C40B 40/06; C40B 30/04

(52) U.S. Cl. 506/9; 435/6.11; 506/16

(57) ABSTRACT

The disclosure provides compositions and methods for determining the likely tumor recurrence in cancer patients by screening CD44, CD166 and/or LGR5 polymorphism in samples isolated from the patient.

### CROSS-REFERENCE TO RELATED APPLICATION

This application claims the benefit under 35 U.S.C. §119(e) of U.S. Provisional Application U.S. Ser. No. 61/486,168, filed May 13, 2011, the contents of which is hereby incorporated by reference into the present disclosure.

### STATEMENT AS TO FEDERALLY SPONSORED RESEARCH OR DEVELOPMENT

This invention wase made with government support under the National Institutes of Health Grant No. 5 P30 CA14089-271. Accordingly, the U.S. Government has certain rights to the invention.

### FIELD OF THE INVENTION

This invention relates to the filed of pharmacogenomics and specifically to the application of genetic polymorphisms to predict outcome of a clinical procedure.

### BACKGROUND

In nature, organisms of the same species usually differ from each other in some aspects, e.g., their appearance. The differences are genetically determined and are referred to as polymorphism. Genetic polymorphism is the occurrence in a population of two or more genetically determined alternative phenotypes due to different alleles. Polymorphism can be observed at the level of the whole individual (phenotype), in variant forms of proteins and blood group substances (biochemical polymorphism), morphological features of chromosomes (chromosomal polymorphism) or at the level of DNA in differences of nucleotides (DNA polymorphism).

Polymorphism also plays a role in determining differences in an individual's response to drugs. Pharmacogenetics and pharmacogenomics are multidisciplinary research efforts to study the relationship between genotype, gene expression profiles, and phenotype, as expressed in variability between individuals in response to or toxicity from drugs. Indeed, it is now known that cancer chemotherapy is limited by the predisposition of specific populations to drug toxicity or poor drug response. For a review of the use of germline polymorphisms in clinical oncology, see Lenz (2004) J. Clin. Oncol. 22(13):2519-2521; Park et al. (2006) Curr. Opin. Pharma. 6(4):337-344; Zhang et al. (2006) Pharma. and Genomics 16(7):475-483 and U.S. Patent Publ. No. 2006/0115827. For a review of pharmacogenetics and pharmacogenomics in therapeutic antibody development for the treatment of cancer, see Yan and Beckman (2005) Biotechniques 39:565-568.

Adjuvant treatment is recommended for patients with stage III and high-risk stage II colon cancer (CC). The risk of tumor recurrence can be significantly reduced by treating these patients with 5-fluorouracil (5-FU)-based chemotherapy. The addition of oxaliplatin to 5-FU-based chemotherapy is now a standard adjuvant treatment for CC, with a higher 5-year disease-free survival (DFS) rate, compared with 5-FU-based treatment alone (73.3% vs 67.4%). (Cunningham et al. (2010) Lancet 375:1030-1047) However, a considerable number of patients will relapse despite adjuvant treatment. It is therefore essential to identify patients who will benefit from adjuvant treatment, sparing others needless toxicity of chemotherapy that will not work. (Tejpar et al. (2010) Oncologist 15:390-404)

Tumor recurrence after curative surgery remains a major obstacle for improving overall cancer survival, which may be in part to the existence of cancer stem cells (CSC). Growing evidence suggests that human cancers are stem cell diseases and only a small subpopulation of cancer cells, endowed with stem cell-like features, might be responsible for tumor initiation, progression and chemoresistance. (Zeki et al. (2011) Nat. Rev. Gastroenterol. Hepatol. 8:90-100) Cancer cells with the properties of stem cells possess the ability to self-renew, to undergo multilineage differentiation, and to survive an adverse tissue microenvironment.

Putative CSC populations have been identified in CC on the basis of the expression of specific markers and their functional properties; however, phenotypic characterization of colon CSCs is still a matter of debate and ongoing research studies. (Todaro et al. (2010) Gastroenterology 138:2151-2162) Ideally, definitive markers should be gene products that are coupled to the function of the stem cell. CSC markers in CC include CD133, CD44, and CD166. (Dalerba et al. (2007) Proc. Natl. Acad. Sci. U.S.A. 104:10158-10163) More recently EpCAM, CD26, Msi-1, CD29, CD24, LGR5 and ALDH1A1 have been added to the list of putative stem cell markers for CC. (Sanders et al. (2010) Front Biosci. 16:1651-1662; Vermeulen et al. (2008) Proc. Natl. Acad. Scie U.S.A. 105:13427-13432) These colon CSC markers are representative of a range of pathways including the Wnt-target genes, cell adhesion molecules, RNA-binding proteins, and detoxifying enzymes, and play distinct roles in a variety of processes including cell differentiation, proliferation, migration, apoptosis, adhesion, lymphocyte homing, angiogenesis and cellular response to chemotherapy. (Saif et al. (2010) Cancer J. 16:196-201) Current therapies target populations of rapidly growing and differentiated tumor cells, but have been shown to lack activity against CSCs. (Todaro et al. (2010) Gastroenterology 138:2151-2162) CSCs therefore may have an important role in tumor recurrence despite adjuvant chemotherapy. Thus far, pre-clinical studies in CC have identified that CSCs are capable of initiating tumor development, however, little is known about the role of CSCs in CC tumor recurrence.

### SUMMARY

In 2010, an estimated 142,570 new cases of colorectal cancer (CRC) and 21,100 new cases of gastric adenocarcinoma (GA) would be diagnosed in the United States. Globally, CRC and GA are responsible for an estimated 529,000 and 700,000 deaths annually, yielding to a case—fatality ratio (CFR) of 0.75 and 0.52, respectively, which is much higher than in other common malignancies like breast cancer (CFR 0.36) and prostate cancer (CFR 0.33). Pathological tumor staging (T stage, N stage) remains the main prognostic determinant for CRC and GA. Patients in early stages who are fortunate enough to undergo surgery, are considered candidates for cure. However, ˜30%-40% of CRC patients and ˜40%-60% of GA patients who underwent surgery followed by adjuvant (radio) chemotherapy will develop recurrence. Consequently, the development of molecular prognostic markers as an adjunct to the conventional clinicopathologic staging is essential in selecting patients at high risk of tumor recurrence, thereby rationalizing treatment strategies and improving outcomes.

Herein, Applicant reports that polymorphisms of certain cancer stem cell genes, e.g., CD44, CD166 and Lgr5, were associated with clinical outcomes, such as tumor recurrence, in colorectal cancer patients. Thus, this disclosure provides compositions, methods and kits for determining the likely tumor recurrence of cancer patients by screening at least one polymorphism of CD44 rs8193 C/T, CD166 rs1157 G/A or LGR5 rs17109926 T/C in samples isolated from the patient.

Thus, in one aspect, the disclosure provides a method for aiding in the determination of or determining whether a cancer patient is likely to experience a longer or shorter time to tumor recurrence, comprising screening a tissue or cell sample isolated from the patient for at least one polymorphism of CD44 rs8193 C/T, CD166 rs1157 G/A or LGR5 rs17109926 T/C, wherein the presence of one or more genotypes of:

(a) (T/T or C/T) for CD44 rs8193 C/T;

(b) (A/A or G/A) for CD166 rs1157 G/A;

(c) (C/C or T/C) for LGR5 rs17109926 T/C; or

(d) (T/T) for LGR5 rs17109926 T/C and (T/T) for CD44 rs8193 C/T; determines that the patient is likely to experience a longer time to tumor recurrence, or the presence of none of genotypes (a)-(d) determines that the patient is likely to experience a shorter time to tumor recurrence.

In one aspect, the patient suffers from at least one cancer of the type of the group of lung cancer, non-small cell lung cancer, breast cancer, head and neck cancer, ovarian cancer, colon cancer, rectal cancer, Stage II or Stage III colon cancer, localized gastric cancer, gastric adenocarcinoma, colorectal cancer, esophageal cancer, gastric cancer, liver cancer, bone cancer, spleen cancer, pancreatic cancer, or gallbladder cancer. In one embodiment, the patient suffers from one or more gastrointestinal cancer. In another embodiment, the gastrointestinal cancer is colon cancer.

The methods are useful in the assistance of an animal, a mammal or yet further a human patient. For the purpose of illustration only, a patient includes but is not limited to a human, a simian, a murine, a bovine, an equine, a porcine, a feline, a canine, or an ovine.

### BRIEF DESCRIPTION OF THE FIGURES

FIGS. 1A through 1C graphically show time to tumor recurrence by (A) CD44 rs8193 C>T, (B) ALCAM rs1157 G>A and (C) LGR5 rs17109924 T>C (see Example 2).

FIG. 2(A) shows a RPart analysis of TTR. The end-nodes of the tree model represent subgroups of low- and high-risk patients based on either a single gene variant or combination of gene variants. Fractions within the end-nodes indicate patients who recurred/total patients with this gene variant profile.

FIG. 2(B) shows TTR by tree model defined subgroups. Node 5 represents a high-risk subgroup based on a specific gene variant profile including LGR5 rs17109924, CD44 rs8193 and ALDH1A1 rs1342024.

### DETAILED DESCRIPTION OF THE DISCLOSURE

Throughout this disclosure, various publications, patents and published patent specifications are referenced by an identifying citation or by an Arabic numeral within parentheses. The complete bibliographic citation for each reference noted by a number within a parenthetical is found in the Reference section, immediately preceding the claims. The disclosures of these publications, patents and published patent specifications are hereby incorporated by reference into the present disclosure to more fully describe the state of the art to which this disclosure pertains.

The practice of the present disclosure employs, unless otherwise indicated, conventional techniques of molecular biology (including recombinant techniques), microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3^{rd} edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5^{th} edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L.A. Herzenberg et al. eds (1996)).

### Definitions

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of when used to define compositions and methods, shall mean excluding other elements of any essential significance to the composition or method. “Consisting of shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

As used herein, the term “screening a cell or tissue sample for a genotype or a polymorphism” intends to identify the genotypes of polymorphic loci of interest in the cell or tissue sample. In one aspect, a polymorphic locus is a single nucleotide polymorphic (SNP) locus. If the allelic composition of a SNP locus is heterozygous, the genotype of the SNP locus will be identified as “X/Y” wherein X and Y are two different nucleotides, e.g., C/T for the CD44 rs8193 C/T SNP. If the allelic composition of a SNP locus is homozygous, the genotype of the SNP locus will be identified as “X/X” wherein X identifies the nucleotide that is present at both alleles, e.g., C/C for the CD44 rs8193 C/T SNP.

The term “genetic marker” refers to an allelic variant of a polymorphic region of a gene of interest and/or the expression level of a gene of interest.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region can be a single nucleotide, the identity of which differs in different alleles.

A “polymorphic gene” refers to a gene having at least one polymorphic region.

The term “genotype” refers to the specific allelic composition of an entire cell or a certain gene and in some aspects a specific polymorphism associated with that gene, whereas the term “phenotype” refers to the detectable outward manifestations of a specific genotype.

The phrase “polymorphisms are determinedincludes screening methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof The antisense strand is the complement of such a nucleic acid, and the encoding sequence can be deduced therefrom.

The term “isolated” or “recombinant” as used herein with respect to nucleic acids, such as DNA or RNA, refers to molecules separated from other DNAs or RNAs, respectively that are present in the natural source of the macromolecule as well as polypeptides. The term “isolated or recombinant nucleic acid” is meant to include nucleic acid fragments which are not naturally occurring as fragments and would not be found in the natural state. The term “isolated” is also used herein to refer to polynucleotides, polypeptides and proteins that are isolated from other cellular proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart.

A “patient” as used herein intends an animal patient, a mammal patient or yet further a human patient. For the purpose of illustration only, a patient includes but is not limited to a simian, a human, a murine, a bovine, an equine, a porcine, a feline, a canine, or an ovine.

“Gastrointestinal cancer” refers to malignant conditions of the gastrointestinal tract. In one aspect, gastrointestinal cancer includes Gastrointestinal stromal tumors (GIST), esophageal cancer, stomach cancer (also called gastric cancer), liver cancer (also called hepatocellular carcinoma, HCC, or hepatoma), gallbladder cancer, pancreatic cancer, colorectal cancer (e.g., called colon cancer, bowel cancer, and rectal cancer) and anal cancer. In one aspect, gastrointestinal cancer includes esophageal cancer, stomach cancer, liver cancer and colorectal cancer. In another aspect, gastrointestinal cancer includes stomach cancer and colorectal cancer.

“Colorectal cancer” or “bowel cancer” refers to a cancer in the colon, rectum, appendix or anus. In one aspect, colorectal cancer include both colon cancer and rectal cancer.

“Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

“Overall Survival” (OS) intends a prolongation in life expectancy as compared to naïve or untreated individuals or patients.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

The term “determine” or “determining” is to associate or affiliate a patient closely to a group or population of patients who likely experience the same or a similar clinical response.

As used herein, the terms “Stage I cancer,” “Stage II cancer,” “Stage III cancer,” and “Stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

A “tumor” is an abnormal growth of tissue resulting from uncontrolled, progressive multiplication of cells and serving no physiological function. A “tumor” is also known as a neoplasm.

As used herein, “surgery” or “surgical resection” refers to surgical removal of a tumor of concern.

“Having a/the same cancer” is used when comparing one patient to another or alternatively, one patient population to another patient population. For example, the two patients or patient population will each have or be suffering from colon cancer.

The term “blood” refers to blood which includes all components of blood circulating in a subject including, but not limited to, red blood cells, white blood cells, plasma, clotting factors, small proteins, platelets and/or cryoprecipitate. This is typically the type of blood which is donated when a human patent gives blood.

A “normal cell corresponding to the tumor tissue type” refers to a normal cell from a same tissue type as the tumor tissue. A non-limiting examples is a normal lung cell from a patient having lung tumor, or a normal colon cell from a patient having colon tumor.

A “blood cell” refers to any of the cells contained in blood. A blood cell is also referred to as an erythrocyte or leukocyte, or a blood corpuscle. Non-limiting examples of blood cells include white blood cells, red blood cells, and platelets.

Plasma is known in the art as the yellow liquid component of blood, in which the blood cells in whole blood are typically suspended. It makes up about 55% of the total blood volume. Blood plasma can be prepared by spinning a tube of fresh blood containing an anti-coagulant in a centrifuge until the blood cells fall to the bottom of the tube. The blood plasma is then poured or drawn off. Blood plasma has a density of approximately 1025 kg/m3, or 1.025 kg/l.

A “native” or “natural” or “wild-type” antigen is a polypeptide, protein or a fragment which contains an epitope and which has been isolated from a natural biological source. It also can specifically bind to an antigen receptor.

### Descriptive Embodiments

There is substantial germline genetic variability within the genes used as markers to identify CSCs, including multiple single nucleotide polymorphisms (SNPs). These common DNA-sequence variations may alter the gene function and/or activity including transcription, translation or splicing, thereby causing inter-individual differences in relation to tumor recurrence capacity and chemoresistance. (Coate et al. (2010) J. Clin. Oncol. 28:4029-4037) Applicant recently tested the impact of common gene variants in the cell surface glycoprotein, CD44, a gastric and colon CSC marker, on clinical outcome of patients with localized gastric adenocarcinoma and found that the minor allele of CD44 rs187116 was significantly associated with decreased time to tumor recurrence (TTR) and overall survival (OS), identifying a “high-risk” patient population based on a germline genetic variant. (Winder et al. (2010) Int. J. Cancer)

In the present study, Applicant investigated 25 germline polymorphisms in a comprehensive panel of genes that have been previously associated with colon CSC to predict tumor recurrence in patients with stage III and high-risk stage II CC. The analyzed CSC genes included CD44, Prominin-1 (CD133), dipeptidyl peptidase-4 (DPP4/CD26), epithelial cell adhesion molecule (EpCAM), activated leukocyte cell adhesion molecule (ALCAM/CD166), musashi homolog-1 (MSI-1), integrin beta-1 (ITGB1/CD29), CD24, leucine-rich repeat containing G protein-coupled receptor-5 (LGRS) and aldehyde dehydrogenase-1 family member A1 (ALDH1A1). To the best of Applicant's knowledge, this is the first study investigating common germline genetic variants in a comprehensive panel of colon CSC genes to predict tumor recurrence. This study was conducted adhering to the reporting recommendations for tumor marker prognostic studies. (McShane et al. (2006) Breast Cancer Res. Treat. 100:229-235; Alonzo (2005) J. Clin. Oncol. 23:9053-9054; McShane et al. (2005) J. Clin. Oncol. 23:9067-9072)

Based on the finding disclosed herein, this disclosure provides diagnostic, prognostic and therapeutic methods, which are based, at least in part, on determination of the identity of a genotype of interest identified herein.

For example, information obtained using the diagnostic assays described herein is useful for determining whether a subject is likely to experience a longer time to tumor recurrence.

A patient's likely clinical outcome following a clinical procedure such as surgery can be expressed in relative terms. For example, a patient having a particular genotype or expression level may experience relatively longer overall survival than a patient or patients not having the genotype or expression level. The patient having the particular genotype or expression level, alternatively, can be considered as likely to survive. Similarly, a patient having a particular genotype or expression level may experience relatively shorter time to tumor recurrence than a patient or patients not having the genotype or expression level. The patient having the particular genotype or expression level, alternatively, can be considered as not likely to suffer tumor recurrence.

It is to be understood that information obtained using the diagnostic assays described herein may be used alone or in combination with other information, such as, but not limited to, genotypes or expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes or expression levels of one or more genes as disclosed herein are used in a panel of genes, each of which contributes to the final diagnosis, prognosis or treatment.

The methods of this disclosure are useful for the diagnosis and prognosis of patients suffering from at least one or more cancer of the group: lung cancer, non-small cell lung cancer, breast cancer, head and neck cancer, ovarian cancer, colon cancer, Stage II or Stage III colon cancer, localized gastric cancer, gastric adenocarcinoma, rectal cancer, colorectal cancer, esophageal cancer, gastric cancer, liver cancer, bone cancer, spleen cancer, pancreatic cancer, or gallbladder cancer.

The methods are useful in the assistance of an animal, a mammal or yet further a human patient. For the purpose of illustration only, a patient includes but is not limited to a human, a simian, a murine, a bovine, an equine, a porcine, a feline, a canine or an ovine.

### Diagnostic Methods

Provided, in one aspect, is a method for aiding in the determination of or determining whether a cancer patient is likely to, or identifying a cancer patient or population of patients that is likely to, experience a longer or shorter time to tumor recurrence, comprising screening a tissue or cell sample isolated from the patient for at least one polymorphism of CD44 rs8193 C/T, CD 166 rs1157 G/A or LGR5 rs17109926 T/C, wherein the presence of one or more genotypes of:

(a) (T/T or C/T) for CD44 rs8193 C/T;

(b) (A/A or G/A) for CD166 rs1157 G/A;

(c) (C/C or T/C) for LGR5 rs17109926 T/C; or

(d) (T/T) for LGR5 rs17109926 T/C and (T/T) for CD44 rs8193 C/T; determines that the patient is likely to experience a longer time to tumor recurrence, or the presence of none of genotypes (a)-(d) determines that the patient is likely to experience a shorter time to tumor recurrence.

In one aspect, the presence of one or more genotypes of:

(a) (T/T or C/T) for CD44 rs8193 C/T;

(b) (A/A or G/A) for CD166 rs1157 G/A;

(c) (C/C or T/C) for LGR5 rs17109926 T/C; or

(d) (T/T) for LGR5 rs17109926 T/C and (T/T) for CD44 rs8193 C/T; determines that the patient is likely to experience a longer time to tumor recurrence, or the presence of none of genotypes (a)-(d) determines that the patient is likely to experience a shorter time to tumor recurrence.

In another aspect, the presence of none of genotypes (a)-(d) determines that the patient is likely to experience a shorter time to tumor recurrence. In one embodiment, a patient likely to experience a shorter time to tumor recurrence is a patient likely to experience a shorter time to tumor recurrence than a patient suffering from a same cancer and having one or more genotypes of (a)-(d).

Provided, in another aspect, is a method for aiding in the determination of or determining whether a cancer patient is likely to experience a longer or shorter time to tumor recurrence, comprising screening a tissue or cell sample isolated from the patient for at least one polymorphism of CD44 rs8193 C/T, CD166 rs1157 G/A or LGR5 rs17109926 T/C. In one aspect, the patient suffers from one or more colorectal cancer. In another aspect, the patient suffers from colon cancer. In another aspect, the colon cancer is a Stage II or III colon cancer. In one aspect, the presence of one or more genotypes of:

(a) (T/T or C/T) for CD44 rs8193 C/T;

(b) (A/A or G/A) for CD166 rs1157 G/A;

(c) (C/C or T/C) for LGR5 rs17109926 T/C; or

(d) (T/T) for LGR5 rs17109926 T/C and (T/T) for CD44 rs8193 C/T; determines that the patient is likely to experience a longer time to tumor recurrence, or the presence of none of genotypes (a)-(d) determines that the patient is likely to experience a shorter time to tumor recurrence.

In one aspect of any of the above methods, the patient suffers from at least one cancer of the type of the group of lung cancer, non-small cell lung cancer, breast cancer, head and neck cancer, ovarian cancer, colon cancer, Stage II or Stage III colon cancer, localized gastric cancer, gastric adenocarcinoma, rectal cancer, colorectal cancer, esophageal cancer, gastric cancer, liver cancer, bone cancer, spleen cancer, pancreatic cancer, or gallbladder cancer. In some embodiments, the patient suffers from one or more gastrointestinal cancer. In one aspect, the gastrointestinal cancer is colorectal cancer, or more particularly, colon cancer. In some embodiments, the colon cancer is stage II or III colon cancer.

In another aspect of any of the above methods, the patient has is an adjuvant patient having received surgical resection. In a further aspect, the patient is a Stage II or Stage III colon cancer patient.

Suitable patient samples in the methods include, but are not limited to a sample comprises, or alternatively consisting essentially of, or yet further consisting of, at least one of blood, a tumor cell, a normal cell adjacent to a tumor, a normal cell corresponding to the tumor tissue type, a blood cell, a peripheral blood lymphocyte, or combinations thereof. The samples can be at least one of an original sample recently isolated from the patient, a fixed tissue, a frozen tissue, a biopsy tissue, a resection tissue, a microdissected tissue, or combinations thereof

Any suitable method for identifying the genotype in the patient sample can be used and the disclosures described herein are not to be limited to these methods. For the purpose of illustration only, the genotype is determined by a method comprising, or alternatively consisting essentially of, or yet further consisting of, polymerase chain reaction analysis (PCR), sequencing analysis, restriction enzyme analysis, mismatch cleavage analysis, single strand conformation polymorphism analysis, denaturing gradient gel electrophoresis, selective oligonucleotide hybridization, selective PCR amplification, selective primer extension, oligonucleotide ligation assay, exonuclease-resistant nucleotide analysis, Genetic Bit Analysis, primer-guided nucleotide incorporation analysis PCR, PCR-restriction fragment length polymorphism (PCR-RFLP), direct DNA sequencing, whole genome sequencing, and/or microarray. These methods as well as equivalents or alternatives thereto are described herein.

The methods are useful in the assistance of an animal, a mammal or yet further a human patient. For the purpose of illustration only, a patient includes but is not limited to a human, a simian, a murine, a bovine, an equine, a porcine, a feline, a canine, or an ovine.

The diagnosis methods described in the present disclosure can provide useful information for optimizing treatment strategy. For example, patients at high risk of tumor recurrence or having a low expectation of survival may be treated with more aggressive therapy and/or sooner. Conversely, those at relatively lower risk of tumor recurrence or having a high expectation of survival may be more suitable for a more conservative and/or less toxic therapy.

### Polymorphic Region

For example, information obtained using the diagnostic assays described herein is useful for determining if a subject will likely, more likely, or less likely to survive or experience tumor recurrence. Based on the prognostic information, a doctor can recommend a therapeutic protocol, useful for treating reducing the malignant mass or tumor in the patient or treat cancer in the individual.

In addition, knowledge of the identity of a particular allele in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. The identity of the genotype or expression patterns of individual patients can then be compared to the genotype or expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

Detection of point mutations or additional base pair repeats can be accomplished by molecular cloning of the specified allele and subsequent sequencing of that allele using techniques known in the art, in some aspects, after isolation of a suitable nucleic acid sample using methods known in the art. Alternatively, the gene sequences can be amplified directly from a genomic DNA preparation from the tumor tissue using PCR, and the sequence composition is determined from the amplified product. As described more fully below, numerous methods are available for isolating and analyzing a subject's DNA for mutations at a given genetic locus such as the gene of interest.

A detection method is allele specific hybridization using probes overlapping the polymorphic site and having about 5, or alternatively 10, or alternatively 20, or alternatively 25, or alternatively 30 nucleotides around the polymorphic region. In another embodiment of the disclosure, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244.

In other detection methods, it is necessary to first amplify at least a portion of the gene of interest prior to identifying the allelic variant. Amplification can be performed, e.g., by PCR and/or LCR, according to methods known in the art. In one embodiment, genomic DNA of a cell is exposed to two PCR primers and amplification for a number of cycles sufficient to produce the required amount of amplified DNA.

Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques known to those of skill in the art. These detection schemes are useful for the detection of nucleic acid molecules if such molecules are present in very low numbers.

In one embodiment, any of a variety of sequencing reactions known in the art can be used to directly sequence at least a portion of the gene of interest and detect allelic variants, e.g., mutations, by comparing the sequence of the sample sequence with the corresponding wild-type (control) sequence. Exemplary sequencing reactions include those based on techniques developed by Maxam and Gilbert (1997) Proc. Natl. Acad. Sci, USA 74:560) or Sanger et al. (1977) Proc. Nat. Acad. Sci, 74:5463). It is also contemplated that any of a variety of automated sequencing procedures can be utilized when performing the subject assays (Biotechniques (1995) 19:448), including sequencing by mass spectrometry (see, for example, U.S. Pat. No. 5,547,835 and International Patent Application Publication Number WO 94/16101, entitled DNA Sequencing by Mass Spectrometry by Koster; U.S. Pat. No. 5,547,835 and international patent application Publication Number WO 94/21822 entitled “DNA Sequencing by Mass Spectrometry Via Exonuclease Degradation” by Koster; U.S. Pat. No. 5,605,798 and International Patent Application No. PCT/US96/03651 entitled DNA Diagnostics Based on Mass Spectrometry by Koster; Cohen et al. (1996) Adv. Chromat. 36:127-162; and Griffin et al. (1993) Appl. Biochem. Bio. 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA Sequencing Employing A Mixed DNA-Polymer Chain Probe” and U.S. Pat. No. 5,571,676 entitled “Method For Mismatch-Directed In Vitro DNA Sequencing.”

In some cases, the presence of the specific allele in DNA from a subject can be shown by restriction enzyme analysis. For example, the specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with 51 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Methods Enzy. 217:286-295. In another embodiment, the control or sample nucleic acid is labeled for detection.

In other embodiments, alterations in electrophoretic mobility is used to identify the particular allelic variant. For example, single strand conformation polymorphism (SSCP) may be used to detect differences in electrophoretic mobility between mutant and wild type nucleic acids (Orita et al. (1989) Proc. Natl. Acad. Sci USA 86:2766; Cotton (1993) Mutat. Res. 285:125-144 and Hayashi (1992) Genet Anal Tech. Appl. 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the identity of the allelic variant is obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing agent gradient to identify differences in the mobility of control and sample DNA (Rosenbaum and Reissner (1987) Biophys. Chem. 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al. (1989) Proc. Natl. Acad. Sci. USA 86:6230 and Wallace et al. (1979) Nucl. Acids Res. 6:3543). Such allele specific oligonucleotide hybridization techniques may be used for the detection of the nucleotide changes in the polymorphic region of the gene of interest. For example, oligonucleotides having the nucleotide sequence of the specific allelic variant are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant disclosure. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren et al. (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson et al. have described a nucleic acid detection assay that combines attributes of PCR and OLA (Nickerson et al. (1990) Proc. Natl. Acad. Sci. (U.S.A.) 87:8923-8927). In this method, PCR is used to achieve the exponential amplification of target DNA, which is then detected using OLA.

Several techniques based on this OLA method have been developed and can be used to detect the specific allelic variant of the polymorphic region of the gene of interest. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996) Nucleic Acids Res. 24: 3728, OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the disclosure, a solution-based method is used for determining the identity of the nucleotide of the polymorphic site. Cohen, D. et al. (French Patent 2,650,840; PCT Appln. No. WO 91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO 91/02087) the method of Goelet, P. et al. supra, is preferably a heterogeneous phase assay, in which the primer or the target molecule is immobilized to a solid phase.

Several primer-guided nucleotide incorporation procedures for assaying polymorphic sites in DNA have been described (Komher, J. S. et al. (1989) Nucl. Acids. Res. 17:7779-7784; Sokolov, B. P. (1990) Nucl. Acids Res. 18:3671; Syvanen, A.-C. et al. (1990) Genomics 8:684-692; Kuppuswamy, M. N. et al. (1991) Proc. Natl. Acad. Sci. (U.S.A.) 88:1143-1147; Prezant, T. R. et al. (1992) Hum. Mutat. 1:159-164; Ugozzoli, L. et al. (1992) GATA 9:107-112; Nyren, P. et al. (1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C. et al. (1993) Amer. J. Hum. Genet. 52:46-59).

If the polymorphic region is located in the coding region of the gene of interest, yet other methods than those described above can be used for determining the identity of the allelic variant. For example, identification of the allelic variant, which encodes a mutated signal peptide, can be performed by using an antibody specifically recognizing the mutant protein in, e.g., immunohistochemistry or immunoprecipitation. Antibodies to the wild-type or signal peptide mutated forms of the signal peptide proteins can be prepared according to methods known in the art.

Often a solid phase support is used as a support capable of binding of a primer, probe, polynucleotide, an antigen or an antibody. Well-known supports include glass, polystyrene, polypropylene, polyethylene, dextran, nylon, amylases, natural and modified celluloses, polyacrylamides, gabbros, and magnetite. The nature of the support can be either soluble to some extent or insoluble for the purposes of the present disclosure. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable supports for binding antibody or antigen, or will be able to ascertain the same by use of routine experimentation.

The methods described herein may be performed, for example, by utilizing pre-packaged diagnostic kits, such as those described below, comprising at least one probe or primer nucleic acid described herein, which may be conveniently used, e.g., to determine whether a subject is likely to experience tumor recurrence following therapy as described herein or has or is at risk of developing disease such as colon cancer.

Sample nucleic acid for use in the above-described diagnostic and prognostic methods can be obtained from any suitable cell type or tissue of a subject. For example, a subject's bodily fluid (e.g. blood) can be obtained by known techniques (e.g., venipuncture). Alternatively, nucleic acid tests can be performed on dry samples (e.g., hair or skin). Diagnostic procedures can also be performed in situ directly upon tissue sections (fixed and/or frozen) of patient tissue obtained from biopsies or resections, such that no nucleic acid purification is necessary. Nucleic acid reagents can be used as probes and/or primers for such in situ procedures (see, for example, Nuovo, G. J. (1992) PCR IN SITU HYBRIDIZATION: PROTOCOLS AND APPLICATIONS, Raven Press, NY).

In addition to methods which focus primarily on the detection of one nucleic acid sequence, profiles can also be assessed in such detection schemes. Fingerprint profiles can be generated, for example, by utilizing a differential display procedure, Northern analysis and/or RT-PCR.

In one embodiment, it is necessary to first amplify at least a portion of the gene of interest prior to identifying the polymorphic region of the gene of interest in a sample. Amplification can be performed, e.g., by PCR and/or LCR, according to methods known in the art. Various non-limiting examples of PCR include the herein described methods.

Allele-specific PCR is a diagnostic or cloning technique is used to identify or utilize single-nucleotide polymorphisms (SNPs). It requires prior knowledge of a DNA sequence, including differences between alleles, and uses primers whose 3′ ends encompass the SNP. PCR amplification under stringent conditions is much less efficient in the presence of a mismatch between template and primer, so successful amplification with an SNP-specific primer signals presence of the specific SNP in a sequence (See, Saiki et al. (1986) Nature 324(6093):163-166 and U.S. Pat. Nos. 5,821,062; 7,052,845 or 7,250,258).

Assembly PCR or Polymerase Cycling Assembly (PCA) is the artificial synthesis of long DNA sequences by performing PCR on a pool of long oligonucleotides with short overlapping segments. The oligonucleotides alternate between sense and antisense directions, and the overlapping segments determine the order of the PCR fragments thereby selectively producing the final long DNA product (See, Stemmer et al. (1995) Gene 164(1):49-53 and U.S. Pat. Nos. 6,335,160; 7,058,504 or 7,323,336)

Asymmetric PCR is used to preferentially amplify one strand of the original DNA more than the other. It finds use in some types of sequencing and hybridization probing where having only one of the two complementary stands is required. PCR is carried out as usual, but with a great excess of the primers for the chosen strand. Due to the slow amplification later in the reaction after the limiting primer has been used up, extra cycles of PCR are required (See, Innis et al. (1988) Proc Natl Acad Sci U.S.A. 85(24):9436-9440 and U.S. Pat. Nos. 5,576,180; 6,106,777 or 7,179,600) A recent modification on this process, known as Linear-After-The-Exponential-PCR (LATE-PCR), uses a limiting primer with a higher melting temperature (Tₘ) than the excess primer to maintain reaction efficiency as the limiting primer concentration decreases mid-reaction (Pierce et al. (2007) Methods Mol. Med. 132:65-85).

Colony PCR uses bacterial colonies, for example E. coli, which can be rapidly screened by PCR for correct DNA vector constructs. Selected bacterial colonies are picked with a sterile toothpick and dabbed into the PCR master mix or sterile water. The PCR is started with an extended time at 95° C. when standard polymerase is used or with a shortened denaturation step at 100° C. and special chimeric DNA polymerase (Pavlov et al. (2006) “Thermostable DNA Polymerases for a Wide Spectrum of Applications: Comparison of a Robust Hybrid TopoTaq to other enzymes”, in Kieleczawa J: DNA Sequencing II: Optimizing Preparation and Cleanup. Jones and Bartlett, pp. 241-257)

Helicase-dependent amplification is similar to traditional PCR, but uses a constant temperature rather than cycling through denaturation and annealing/extension cycles. DNA Helicase, an enzyme that unwinds DNA, is used in place of thermal denaturation (See, Myriam et al. (2004) EMBO reports 5(8):795-800 and U.S. Pat. No. 7,282,328).

Hot-start PCR is a technique that reduces non-specific amplification during the initial set up stages of the PCR. The technique may be performed manually by heating the reaction components to the melting temperature (e.g., 95° C.) before adding the polymerase (Chou et al. (1992) Nucleic Acids Research 20:1717-1723 and U.S. Pat. Nos. 5,576,197 and 6,265,169). Specialized enzyme systems have been developed that inhibit the polymerase's activity at ambient temperature, either by the binding of an antibody (Sharkey et al. (1994) Bio/Technology 12:506-509) or by the presence of covalently bound inhibitors that only dissociate after a high-temperature activation step. Hot-start/cold-finish PCR is achieved with new hybrid polymerases that are inactive at ambient temperature and are instantly activated at elongation temperature.

Intersequence-specific (ISSR) PCR method for DNA fingerprinting that amplifies regions between some simple sequence repeats to produce a unique fingerprint of amplified fragment lengths (Zietkiewicz et al. (1994) Genomics 20(2):176-83).

Inverse PCR is a method used to allow PCR when only one internal sequence is known. This is especially useful in identifying flanking sequences to various genomic inserts. This involves a series of DNA digestions and self ligation, resulting in known sequences at either end of the unknown sequence (Ochman et al. (1988) Genetics 120:621-623 and U.S. Pat. Nos. 6,013,486; 6,106,843 or 7,132,587).

Ligation-mediated PCR uses small DNA linkers ligated to the DNA of interest and multiple primers annealing to the DNA linkers; it has been used for DNA sequencing, genome walking, and DNA footprinting (Mueller et al. (1988) Science 246:780-786).

Methylation-specific PCR (MSP) is used to detect methylation of CpG islands in genomic DNA (Herman et al. (1996) Proc Natl Acad Sci U.S.A. 93(13):9821-9826 and U.S. Pat. Nos. 6,811,982; 6,835,541 or 7,125,673). DNA is first treated with sodium bisulfite, which converts unmethylated cytosine bases to uracil, which is recognized by PCR primers as thymine. Two PCRs are then carried out on the modified DNA, using primer sets identical except at any CpG islands within the primer sequences. At these points, one primer set recognizes DNA with cytosines to amplify methylated DNA, and one set recognizes DNA with uracil or thymine to amplify unmethylated DNA. MSP using qPCR can also be performed to obtain quantitative rather than qualitative information about methylation.

Multiplex Ligation-dependent Probe Amplification (MLPA) permits multiple targets to be amplified with only a single primer pair, thus avoiding the resolution limitations of multiplex PCR (see below).

Multiplex-PCR uses of multiple, unique primer sets within a single PCR mixture to produce amplicons of varying sizes specific to different DNA sequences (See, U.S. Pat. Nos. 5,882,856; 6,531,282 or 7,118,867). By targeting multiple genes at once, additional information may be gained from a single test run that otherwise would require several times the reagents and more time to perform Annealing temperatures for each of the primer sets must be optimized to work correctly within a single reaction, and amplicon sizes, i.e., their base pair length, should be different enough to form distinct bands when visualized by gel electrophoresis.

Nested PCR increases the specificity of DNA amplification, by reducing background due to non-specific amplification of DNA. Two sets of primers are being used in two successive PCRs. In the first reaction, one pair of primers is used to generate DNA products, which besides the intended target, may still consist of non-specifically amplified DNA fragments. The product(s) are then used in a second PCR with a set of primers whose binding sites are completely or partially different from and located 3′ of each of the primers used in the first reaction (See, U.S. Pat. Nos. 5,994,006; 7,262,030 or 7,329,493). Nested PCR is often more successful in specifically amplifying long DNA fragments than conventional PCR, but it requires more detailed knowledge of the target sequences.

Overlap-extension PCR is a genetic engineering technique allowing the construction of a DNA sequence with an alteration inserted beyond the limit of the longest practical primer length.

Quantitative PCR (Q-PCR), also known as RQ-PCR, QRT-PCR and RTQ-PCR, is used to measure the quantity of a PCR product following the reaction or in real-time. See, U.S. Pat. Nos. 6,258,540; 7,101,663 or 7,188,030. Q-PCR is the method of choice to quantitatively measure starting amounts of DNA, cDNA or RNA. Q-PCR is commonly used to determine whether a DNA sequence is present in a sample and the number of its copies in the sample. The method with currently the highest level of accuracy is digital PCR as described in U.S. Pat. No. 6,440,705; U.S. Publication No. 2007/0202525; Dressman et al. (2003) Proc. Natl. Acad. Sci USA 100(15):8817-8822 and Vogelstein et al. (1999) Proc. Natl. Acad. Sci. USA. 96(16):9236-9241. More commonly, RT-PCR refers to reverse transcription PCR (see below), which is often used in conjunction with Q-PCR. QRT-PCR methods use fluorescent dyes, such as Sybr Green, or fluorophore-containing DNA probes, such as TaqMan, to measure the amount of amplified product in real time.

Reverse Transcription PCR (RT-PCR) is a method used to amplify, isolate or identify a known sequence from a cellular or tissue RNA (See, U.S. Pat. Nos. 6,759,195; 7,179,600 or 7,317,111). The PCR is preceded by a reaction using reverse transcriptase to convert RNA to cDNA. RT-PCR is widely used in expression profiling, to determine the expression of a gene or to identify the sequence of an RNA transcript, including transcription start and termination sites and, if the genomic DNA sequence of a gene is known, to map the location of exons and introns in the gene. The 5′ end of a gene (corresponding to the transcription start site) is typically identified by an RT-PCR method, named Rapid Amplification of cDNA Ends (RACE-PCR).

Thermal asymmetric interlaced PCR (TAIL-PCR) is used to isolate unknown sequence flanking a known sequence. Within the known sequence TAIL-PCR uses a nested pair of primers with differing annealing temperatures; a degenerate primer is used to amplify in the other direction from the unknown sequence (Liu et al. (1995) Genomics 25(3):674-81).

Touchdown PCR a variant of PCR that aims to reduce nonspecific background by gradually lowering the annealing temperature as PCR cycling progresses. The annealing temperature at the initial cycles is usually a few degrees (3-5° C.) above the Tₘ of the primers used, while at the later cycles, it is a few degrees (3-5 ° C.) below the primer Tₘ. The higher temperatures give greater specificity for primer binding, and the lower temperatures permit more efficient amplification from the specific products formed during the initial cycles (Don et al. (1991) Nucl Acids Res 19:4008 and U.S. Pat. No. 6,232,063).

In one embodiment of the disclosure, probes are labeled with two fluorescent dye molecules to form so-called “molecular beacons” (Tyagi, S. and Kramer, F. R. (1996) Nat. Biotechnol. 14:303-8). Such molecular beacons signal binding to a complementary nucleic acid sequence through relief of intramolecular fluorescence quenching between dyes bound to opposing ends on an oligonucleotide probe. The use of molecular beacons for genotyping has been described (Kostrikis, L. G. (1998) Science 279:1228-9) as has the use of multiple beacons simultaneously (Marras, S. A. (1999) Genet. Anal. 14:151-6). A quenching molecule is useful with a particular fluorophore if it has sufficient spectral overlap to substantially inhibit fluorescence of the fluorophore when the two are held proximal to one another, such as in a molecular beacon, or when attached to the ends of an oligonucleotide probe from about 1 to about 25 nucleotides.

Labeled probes also can be used in conjunction with amplification of a gene of interest. (Holland et al. (1991) Proc. Natl. Acad. Sci. 88:7276-7280). U.S. Pat, No. 5,210,015 by Gelfand et al. describe fluorescence-based approaches to provide real time measurements of amplification products during PCR. Such approaches have either employed intercalating dyes (such as ethidium bromide) to indicate the amount of double-stranded DNA present, or they have employed probes containing fluorescence-quencher pairs (also referred to as the “Taq-Man” approach) where the probe is cleaved during amplification to release a fluorescent molecule whose concentration is proportional to the amount of double-stranded DNA present. During amplification, the probe is digested by the nuclease activity of a polymerase when hybridized to the target sequence to cause the fluorescent molecule to be separated from the quencher molecule, thereby causing fluorescence from the reporter molecule to appear. The Taq-Man approach uses a probe containing a reporter molecule--quencher molecule pair that specifically anneals to a region of a target polynucleotide containing the polymorphism.

Probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the disclosure also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley, S. O. et al. (1999) Nucleic Acids Res. 27:4830-4837.

This disclosure also provides for a prognostic panel of genetic markers selected from, but not limited to the genetic polymorphisms identified herein. The prognostic panel comprises probes or primers or microarrays that can be used to amplify and/or for determining the molecular structure of the polymorphisms identified herein. The probes or primers can be attached or supported by a solid phase support such as, but not limited to a gene chip or microarray. The probes or primers can be detectably labeled. This aspect of the disclosure is a means to identify the genotype of a patient sample for the genes of interest identified above.

In one aspect, the panel contains the herein identified probes or primers as wells as other probes or primers. In a alternative aspect, the panel includes one or more of the above noted probes or primers and others. In a further aspect, the panel consist only of the above-noted probes or primers.

Primers or probes can be affixed to surfaces for use as “gene chips” or “microarray.” Such gene chips or microarrays can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the disclosure also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarray” and similar technologies are know in the art. Examples of such include, but are not limited to LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarraying system that can dispense from 12 to 64 spots onto multiple glass slides (Intelligent Bio-Instruments); Molecular Biology Workstation and NanoChip (Nanogen, Inc.); a microfluidic glass chip (Orchid biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu Rev. Biomed. Eng. 4:129-153. Examples of “Gene chips” or a “microarray” are also described in U.S. Patent Publ. Nos. 2007/0111322, 2007/0099198, 2007/0084997, 2007/0059769 and 2007/0059765 and U.S. Pat. Nos. 7,138,506, 7,070,740, and 6,989,267.

In one aspect, “gene chips” or “microarrays” containing probes or primers for the gene of interest are provided alone or in combination with other probes and/or primers. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers may be detectably labeled thereby identifying the polymorphism in the gene(s) of interest. Alternatively, a chemical or biological reaction may be used to identify the probes or primers which hybridized with the DNA or RNA of the gene(s) of interest. The genetic profile of the patient is then determined with the aid of the aforementioned apparatus and methods.

### Nucleic Acids

In one aspect, the nucleic acid sequences of the gene of interest, or portions thereof, can be the basis for probes or primers, e.g., in methods for determining expression level of the gene of interest or the allelic variant of a polymorphic region of a gene of interest identified in the experimental section below. Thus, they can be used in the methods of the disclosure to determine which therapy is most likely to treat an individual's cancer.

The methods of the disclosure can use nucleic acids isolated from vertebrates. In one aspect, the vertebrate nucleic acids are mammalian nucleic acids. In a further aspect, the nucleic acids used in the methods of the disclosure are human nucleic acids.

Primers for use in the methods of the disclosure are nucleic acids which hybridize to a nucleic acid sequence which is adjacent to the region of interest or which covers the region of interest and is extended. A primer can be used alone in a detection method, or a primer can be used together with at least one other primer or probe in a detection method. Primers can also be used to amplify at least a portion of a nucleic acid. Probes for use in the methods of the disclosure are nucleic acids which hybridize to the gene of interest and which are not further extended. For example, a probe is a nucleic acid which hybridizes to the gene of interest, and which by hybridization or absence of hybridization to the DNA of a subject will be indicative of the identity of the allelic variant of the expression levels of the gene of interest. Primers and/or probes for use in the methods can be provided as isolated single stranded oligonucleotides or alternatively, as isolated double stranded oligonucleotides.

In one embodiment, primers comprise a nucleotide sequence which comprises a region having a nucleotide sequence which hybridizes under stringent conditions to about: 6, or alternatively 8, or alternatively 10, or alternatively 12, or alternatively 25, or alternatively 30, or alternatively 40, or alternatively 50, or alternatively 75 consecutive nucleotides of the gene of interest.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the disclosure will hybridize selectively to nucleotide sequences located about 100 to about 1000 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified.

Yet other preferred primers of the disclosure are nucleic acids which are capable of selectively hybridizing to the gene. Thus, such primers can be specific for the gene of interest sequence, so long as they have a nucleotide sequence which is capable of hybridizing to the gene of interest.

The probe or primer may further comprises a label attached thereto, which, e.g., is capable of being detected, e.g. the label group is selected from amongst radioisotopes, fluorescent compounds, enzymes, and enzyme co-factors.

Additionally, the isolated nucleic acids used as probes or primers may be modified to become more stable. Exemplary nucleic acid molecules which are modified include phosphoramidate, phosphothioate and methylphosphonate analogs of DNA (see also U.S. Pat. Nos. 5,176,996; 5,264,564 and 5,256,775).

The nucleic acids used in the methods of the disclosure can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane. See, e.g., Letsinger et al. (1989) Proc. Natl. Acad. Sci. U.S.A. 86:6553-6556; Lemaitre et al. (1987) Proc. Natl. Acad. Sci. 84:648-652; and PCT Publ. No. WO 88/09810, published Dec. 15, 1988), hybridization-triggered cleavage agents, (see, e.g., Krol et al. (1988) BioTechniques 6:958-976) or intercalating agents (see, e.g., Zon (1988) Pharm. Res. 5:539-549. To this end, the nucleic acid used in the methods of the disclosure may be conjugated to another molecule, e.g., a peptide, hybridization triggered cross-linking agent, transport agent, hybridization-triggered cleavage agent, etc.

The isolated nucleic acids used in the methods of the disclosure can also comprise at least one modified sugar moiety selected from the group including but not limited to arabinose, 2-fluoroarabinose, xylulose, and hexose or, alternatively, comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The nucleic acids, or fragments thereof, to be used in the methods of the disclosure can be prepared according to methods known in the art and described, e.g., in Sambrook et al. (2001) supra. For example, discrete fragments of the DNA can be prepared and cloned using restriction enzymes. Alternatively, discrete fragments can be prepared using the Polymerase Chain Reaction (PCR) using primers having an appropriate sequence under the manufacturer's conditions, (described above).

Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988) Nucl. Acids Res. 16:3209, methylphosphonate oligonucleotides can be prepared by use of controlled pore glass polymer supports. Sarin et al. (1988) Proc. Natl. Acad. Sci. U.S.A. 85:7448-7451.

### Methods of Treatment

The diagnostic methods described in the present disclosure can provide useful information for optimizing treatment strategy. For example, patients at high risk of tumor recurrence or having a low expectation of survival may be treated with more aggressive therapy and/or sooner. Conversely, those at relatively lower risk of tumor recurrence or having a high expectation of survival may be more suitable for a more conservative and/or less toxic therapy.

The following therapies are available for cancer patients, such as colorectal cancer patients, to prevent or reduce tumor recurrence: 5-fluorouracil (5-FU), capecitabine (Xeloda), Leucovorin (LV, folinic Acid), Oxaliplatin (Eloxatin), the combination of infusional 5-fluorouracil, leucovorin, and oxaliplatin (FOLFOX) with bevacizumab or infusional 5-fluorouracil, leucovorin, and irinotecan (FOLFIRI) with bevacizumab, Tegafur-uracil, Irinotecan (Camptosar), Oxaliplatin (Eloxatin), Bevacizumab (Avastin), Cetuximab (Erbitux), Panitumumab (Vectibix), Bortezomib (Velcade), Oblimersen (Genasense, G3139), Gefitinib and erlotinib (Tarceva) or Topotecan (Hycamtin). Therapeutic and adverse effects of these therapies have been studied. The therapy can further comprise radiation therapy. The diagnostic methods provided in the present disclosure, therefore, are useful in optimal selection of these therapies.

Accordingly, this disclosure also provides methods for treating a cancer patient. In one embodiment, a cancer patient, which is predicted to experience a relatively shorter time to tumor recurrence or relatively more likely to experience tumor recurrence, is treated with a more aggressive therapy such as a therapy at a higher dose or a higher frequency. In another embodiment, a cancer patient, which is predicted to experience a relatively longer time to tumor recurrence or relatively less likely to experience tumor recurrence, is treated with a safer therapy or a therapy causing less adverse effects, such as a therapy at a lower dose or a lower frequency.

The methods are useful to treat patients that include but are not limited to animals, such as mammals which can include simians, ovines, bovines, murines, canines, equines, felines, canines, and humans.

The therapies can be administered by any suitable formulation. Accordingly, a formulation comprising the necessary therapy is further provided herein. The formulation can further comprise one or more preservatives or stabilizers. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1., 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, and 1.0%).

The chemotherapeutic agents or drugs can be administered as a composition. A “composition” typically intends a combination of the active agent and another carrier, e.g., compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this disclosure, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term carrier further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-.quadrature.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional proviso that they be acceptable for use in vivo. For examples of carriers, stabilizers and adjuvants, see Martin REMINGTON'S PHARM. SCI., 15th Ed. (Mack Publ. Co., Easton (1975) and Williams & Williams, (1995), and in the “PHYSICIAN'S DESK REFERENCE”, 52^{nd} ed., Medical Economics, Montvale, N.J. (1998).

Many combination chemotherapeutic regimens are known to the art, such as combinations of platinum compounds and taxanes, e.g. carboplatin/paclitaxel, capecitabine/docetaxel, the “Cooper regimen”, fluorouracil-levamisole, fluorouracil-leucovorin, fluorouracil/oxaliplatin, methotrexate-leucovorin, and the like.

Combinations of chemotherapies and molecular targeted therapies, biologic therapies, and radiation therapies are also well known to the art; including therapies such as trastuzumab plus paclitaxel, alone or in further combination with platinum compounds such as oxaliplatin, for certain breast cancers, and many other such regimens for other cancers; and the “Dublin regimen” 5-fluorouracil IV over 16 hours on days 1-5 and 75 mg/m² cisplatin IV or oxaliplatin over 8 hours on day 7, with repetition at 6 weeks, in combination with 40 Gy radiotherapy in 15 fractions over the first 3 weeks) and the “Michigan regimen” (fluorouracil plus cisplatin or oxaliplatin plus vinblastine plus radiotherapy), both for esophageal cancer, and many other such regimens for other cancers, including colorectal cancer.

In another aspect of the disclosure, the method for treating a patient further comprises, or alternatively consists essentially of, or yet further consists of surgical resection of a metastatic or non-metastatic solid malignant tumor and, in some aspects, in combination with radiation. Methods for treating these tumors as Stage I, Stage II, Stage III, or Stage IV by surgical resection and/or radiation are known to one skilled in the art. Guidelines describing methods for treatment by surgical resection and/or radiation can be found at the National Comprehensive Cancer Network's web site, nccn.org, last accessed on May 27, 2008.

The disclosure provides an article of manufacture, comprising packaging material and at least one vial comprising a solution of the chemotherapy as described herein and/or or at least one antibody or its biological equivalent with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The disclosure further comprises an article of manufacture, comprising packaging material, a first vial comprising the chemotherapy and/or at least one lyophilized antibody or its biological equivalent and a second vial comprising an aqueous diluent of prescribed buffer or preservative, wherein said packaging material comprises a label that instructs a patient to reconstitute the therapeutic in the aqueous diluent to form a solution that can be held over a period of twenty-four hours or greater.

Chemotherapeutic formulations of the present disclosure can be prepared by a process which comprises mixing at least one antibody or biological equivalent and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing of the antibody and preservative in an aqueous diluent is carried out using conventional dissolution and mixing procedures. For example, a measured amount of at least one antibody in buffered solution is combined with the desired preservative in a buffered solution in quantities sufficient to provide the antibody and preservative at the desired concentrations. Variations of this process would be recognized by one of skill in the art, e.g., the order the components are added, whether additional additives are used, the temperature and pH at which the formulation is prepared, are all factors that can be optimized for the concentration and means of administration used.

The compositions and formulations can be provided to patients as clear solutions or as dual vials comprising a vial of lyophilized antibody that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available. Recognized devices comprising these single vial systems include those pen-injector devices for delivery of a solution such as BD Pens, BD Autojectore, Humaject® NovoPen®, B-D®Pen, AutoPen®, and OptiPen®, GenotropinPen®, Genotronorm Pen®, Humatro Pen®, Reco-Pen®, Roferon Pen®, Biojector®, iject®, J-tip Needle-Free Injector®, Intraject®, Medi-Ject®, e.g., as made or developed by Becton Dickensen (Franklin Lakes, N.J. available at bectondickenson.com), Disetronic (Burgdorf, Switzerland, available at disetronic.com; Bioject, Portland, Oreg. (available at bioject.com); National Medical Products, Weston Medical (Peterborough, UK, available at weston-medical.com), Medi-Ject Corp (Minneapolis, Minn., available at mediject.com).

Various delivery systems are known and can be used to administer a chemotherapeutic agent of the disclosure, e.g., encapsulation in liposomes, microparticles, microcapsules, expression by recombinant cells, receptor-mediated endocytosis. See e.g., Wu and Wu (1987) J. Biol. Chem. 262:4429-4432 for construction of a therapeutic nucleic acid as part of a retroviral or other vector, etc. Methods of delivery include but are not limited to intra-arterial, intra-muscular, intravenous, intranasal and oral routes. In a specific embodiment, it may be desirable to administer the pharmaceutical compositions of the disclosure locally to the area in need of treatment; this may be achieved by, for example, and not by way of limitation, local infusion during surgery, by injection or by means of a catheter.

The agents identified herein as effective for their intended purpose can be administered to subjects or individuals identified by the methods herein as suitable for the therapy. Therapeutic amounts can be empirically determined and will vary with the pathology being treated, the subject being treated and the efficacy and toxicity of the agent.

Also provided is a therapy or a medicament comprising an effective amount of a chemotherapeutic as described herein for treatment of a human cancer patient having the appropriate expression level of the gene of interest as identified in the experimental examples. Further provided is a therapy comprising a platinum drug, or alternatively a platinum drug therapy, for use in treating a human cancer patient having the appropriate expression level of the gene of interest as identified in the experimental examples.

Methods of administering pharmaceutical compositions are well known to those of ordinary skill in the art and include, but are not limited to, oral, microinjection, intravenous or parenteral administration. The compositions are intended for topical, oral, or local administration as well as intravenously, subcutaneously, or intramuscularly. Administration can be effected continuously or intermittently throughout the course of the treatment. Methods of determining the most effective means and dosage of administration are well known to those of skill in the art and will vary with the cancer being treated and the patient and the subject being treated. Single or multiple administrations can be carried out with the dose level and pattern being selected by the treating physician.

### Kits

Also provided is a kit for use in aiding in the determination of or determining whether a cancer patient is likely to, or identifying a patient or population of patients that is likely to, experience a longer or shorter time to tumor recurrence, comprising, or alternatively consisting essentially of, or yet alternatively consisting of, suitable primers or probes or a microarray for screening a tissue or cell sample isolated from the patient for at least one polymorphism of CD44 rs8193 C/T, CD166 rs1157 G/A or LGR5 rs17109926 T/C, and instructions for use therein.

In one aspect of any of the kits, the patient suffers from one or more cancer selected from lung cancer, non-small cell lung cancer, breast cancer, head and neck cancer, ovarian cancer, colon cancer, Stage II or Stage III colon cancer, localized gastric cancer, gastric adenocarcinoma, rectal cancer, colorectal cancer, esophageal cancer, gastric cancer, liver cancer, bone cancer, spleen cancer, pancreatic cancer, or gallbladder cancer.

Suitable patient samples for the methods as described herein, the sample comprises, or alternatively consisting essentially of, or yet further consisting of, at least one of blood, plasma, a tumor cell, a normal cell adjacent to a tumor, a normal cell corresponding to the tumor tissue type, a blood cell, a peripheral blood lymphocyte, or combinations thereof. The samples can be any one or more of a fixed tissue, a frozen tissue, a biopsy tissue, a resection tissue, a microdissected tissue, or combinations thereof

Any suitable method for identifying the genotype in the patient sample can be used and the disclosures described herein are not to be limited to these methods. For the purpose of illustration only, the genotype is determined by a method comprising, or alternatively consisting essentially of, or yet further consisting of, polymerase chain reaction analysis (PCR), sequencing analysis, restriction enzyme analysis, mismatch cleavage analysis, single strand conformation polymorphism analysis, denaturing gradient gel electrophoresis, selective oligonucleotide hybridization, selective PCR amplification, selective primer extension, oligonucleotide ligation assay, exonuclease-resistant nucleotide analysis, Genetic Bit Analysis, primer-guided nucleotide incorporation analysis PCR, PCR-restriction fragment length polymorphism (PCR-RFLP), direct DNA sequencing, whole genome sequencing, and/or microarray. These methods as well as equivalents or alternatives thereto are described herein or known in the art.

The methods are useful in the assistance of an animal, a mammal or yet further a human patient. For the purpose of illustration only, a patient includes but is not limited to a simian, a murine, a bovine, an equine, a porcine, a feline, a canine, or an ovine.

As set forth herein, the disclosure provides diagnostic methods for determining the polymorphic region of the gene of interest. In some embodiments, the methods use probes or primers or microarrays comprising nucleotide sequences which are complementary to the gene of interest. Accordingly, the disclosure provides kits for performing these methods as well as instructions for carrying out the methods of this disclosure such as collecting tissue and/or performing the screen, and/or analyzing the results. These can be used alone or in combination with other suitable chemotherapy or biological therapy.

The kit can comprise at least one probe or primer which is capable of specifically hybridizing to the gene of interest and instructions for use. The kits preferably comprise at least one of the above described nucleic acids. Preferred kits for amplifying at least a portion of the gene of interest comprise two primers, at least one of which is capable of hybridizing to the allelic variant sequence. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the disclosure comprise at least one reagent necessary to perform the assay. For example, the kit can comprise an enzyme. Alternatively the kit can comprise a buffer or any other necessary reagent.

Conditions for incubating a nucleic acid probe with a test sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present disclosure. Examples of such assays can be found in Chard, T. (1986) AN INTRODUCTION TO RADIOIMMUNOASSAY AND RELATED TECHNIQUES Elsevier Science Publishers, Amsterdam, The Netherlands; Bullock, G. R. et al., TECHNIQUES IN IMMUNOCYTOCHEMISTRY Academic Press, Orlando, Fla. Vol. 1 (1982), Vol. 2 (1983), Vol. 3 (1985); Tijssen, P. (1985) PRACTICE AND THEORY OF IMMUNOASSAYS: LABORATORY TECHNIQUES IN BIOCHEMISTRY AND MOLECULAR BIOLOGY, Elsevier Science Publishers, Amsterdam, The Netherlands.

The test samples used in the diagnostic kits include cells, protein or membrane extracts of cells, or biological fluids such as sputum, blood, serum, plasma, or urine. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region of the gene of interest.

As amenable, these suggested kit components may be packaged in a manner customary for use by those of skill in the art. For example, these suggested kit components may be provided in solution or as a liquid dispersion or the like.

### Other Uses for the Nucleic Acids of the Disclosure

The identification of the polymorphic region or the expression level of the gene of interest can also be useful for identifying an individual among other individuals from the same species. For example, DNA sequences can be used as a fingerprint for detection of different individuals within the same species. Thompson, J. S. and Thompson, eds., (1991) GENETICS IN MEDICINE, W B Saunders Co., Philadelphia, Pa. This is useful, e.g., in forensic studies.

The disclosure now being generally described, it will be more readily understood by reference to the following example which is included merely for purposes of illustration of certain aspects and embodiments of the present disclosure, and are not intended to limit the disclosure.

### EXPERIMENTAL DETAILS

### Example 1

This example examines whether genetic variants of certain cancer stem cell genes are associated with tumor recurrence.

Methods: Either blood or FFPE tissue specimens were obtained from 234 patients (107 females and 127 males; median age 59 yrs (range 22-87 yrs)) with stage II (105 pts) or III (129 pts) colon cancer at the University of Southern California. The median follow-up was 4.4 years. Twenty-six SNPs in genes of cancer stem cell markers (CD44, CD133, CD26, EpCAM, CD166, Msi-1, CD29, CD24, LGR5 and ALDH1) were determined by PCR-RFLP or direct DNA-sequencing. Primary endpoint of the study was time to tumor recurrence (TTR). This study was conducted adhering to the reporting recommendations for tumor marker prognostic studies (REMARK). Primers for the PCR amplification are listed in Table 1.

TABLE 1
Primer sequences

| | | | | |
|---|---|---|---|---|
| Gene/SNP | Region | Primer F (SEQ ID NO:) | Primer R (SEQ ID NO:) | Enzyme |

CD44

| | | | | |
|---|---|---|---|---|
| rs8193 C/T | 3UTR | CAGGGTTAATAGGGCCT | GAAAAATTTCTAGAGGG | BsrDI |
| | | GGT (1) | GGTCTG (2) | |
| rs187115 T/C | Intron | CTCTGTCTCTCCTGCCC | GCTAATTCAAATGCTTG | DS |
| | | AAT (3) | GTTG (4) | |
| rs187116 | Intron | AGGTGGTTGGAGATCAC | CTTTCGCAAGAACCACT | MspI |
| G/A | | CTG (5) | TCC (6) | |
| rs4755392 | Downstream | TGGGTAATTTAGAGGAA | ACACATCACTCATAGAA | DS |
| T/A | | CAAAGTCA (7) | AACCAGA (8) | |
| rs7116432 | 3UTR | CATCGTCTTCTTGCTGT | GGTCTTGGTTCAGGTAG | NlaIII |
| G/A | | TAGGA (9) | GGAGA (10) | |

CD133

| | | | | |
|---|---|---|---|---|
| rs3130 C/T | 3UTR | CTGCAATCTGCACAT | TGCATGAAAGCACAAGG | Ecor-1 |
| | | GAA AAG (11) | T AAA (12) | |
| rs2240688 | 3UTR | GTCAGATGG AGT TAC | AATCCATTTTCACTAAA | CVIKI-1 |
| T/G | | GCAGGT (13) | A GTGTGT G (14) | |
| rs2286455 | Splice Site | ACGCCTCTTTGGTCTCC | TCCATCCCAAGTCCCTT | MbO-1 |
| C/T | | TTG (15) | TAG (16) | |

CD26

| | | | | |
|---|---|---|---|---|
| rs2300757 | Intron | CTGCAGAGCCCTGTAGC | TGACGATATGGGCTTGT | TFi I |
| G/C | | C (17) | GAG (18) | (G cut) |
| rs1014444 | Intron | TTAAGAAGGGGGAGTTG | GGTGCTATGGGAAAT | Alu I (A |
| A/G | | TGG (19) | GCA AA (20) | cut; 2x!) |
| rs2268894 | Intron | CGACTCCACAGCATCTC | TCTCCTCCTCTTATCAG | DS |
| C/T | | T GA (21) | CTCTCTC (22) | |

EPCAM

| | | | | |
|---|---|---|---|---|
| rs17036526 | Splice site | GCCACGTGATCATTCTC | TTCCTTTGGGAAAGATC | DdeI |
| G/C | | TAGC (23) | AAAAA (24) | (C cut) |
| rs1126497 | Non- | CCAGAACAATGATGGGC | CACTCGCTCAGAGCAGG | NiAIII |
| T/C | synonymou | TTT (25) | TTA (26) | (T cut) |
| | s coding | | | |
| rs1421 T/C | 3UTR | GGGAAATAGCAAATGGA | ATTGGTAAAGCCAGTTT | DS |
| | | CACA (27) | CAAGC (28) | |

CD166

| | | | | |
|---|---|---|---|---|
| rs6437585 | 5UTR | GGAGGGAGGAGGAGTTG | CGTCTTCTCCCAGACAC | Cac8I |
| C/T | | G (29) | ACC (30) | (C cut) |
| rs1044240 | Non- | GCACAGAGTAATTCGGT | TTGCCATTCCCTAAGCA | DS |
| A/G | synonymous | ACTTGA (31) | TTT (32) | |
| | coding | | | |
| rs1044243 | Non- | AATCATCTGACATTTTG | AGCTGTTGAAGCAATCA | DS |
| C/T | synonymous | CCTCT (33) | TGC (34) | |
| | coding | | | |
| rs1157 G/A | 3UTR | CCAAAAACAGCTGTCAG | GGCTGCCATTAAACAAG | MspI |
| | | AACC (35) | TAAGC (36) | (G cut) |

Msi-1

| | | | | |
|---|---|---|---|---|
| rs2522137 | 3UTR | CCTGGTGCCCACTCATT | CGACACTGCTGGACAGG | DS |
| A/C | | G (37) | AA (38) | |

CD29

| | | | | |
|---|---|---|---|---|
| rs2153875 | Splice site | CAGTGTTGTGGGATTTG | GGGTAACTGATAATTTT | BfaI |
| G/T | | CAC (39) | TCTCACTTTT (40) | (G cut) |

Cd24

| | | | | |
|---|---|---|---|---|
| rs8734 C/T | Missense | CCTCCCAGAGTACTTCC | ACCACGAAGAGACTGGC | AciI |
| | | AACTC (41) | TGT (42) | (C cut) |
| rs3838646- | 3UTR | CAAATGTGGCTATTCTG | GGCAAAAATGTAAAGGA | BsrI |
| C/A | | ATCCA (43) | GTCAAA (44) | |

LGR5

| | | | | |
|---|---|---|---|---|
| rs17109924 | Non- | GGTTGCCATGTCATTGG | AGGGCACAGAGCAAAAT | DS |
| T/C | synonymous | TTT (45) | GAT (46) | |
| | coding | | | |
| rs17109926 | Downstream | GAAAAGGCTGAAAACCT | TTTTTGATCTGGGCTCA | DS |
| G/A | | CTTGA (47) | CTT (48) | |

ALDH1

| | | | | |
|---|---|---|---|---|
| rs13959 C/T | Synonymous | GATAATACTCACCGCCA | GGATGTTGACAAGGCAG | Hypch4III |
| | coding | GCAG (49) | TGA (50) | (T cut) |
| rs1342024 | Upstream | TCCATGAAGCACAAAAC | GTTTGGGGCACTCCTTC | DS |
| G/C | | ACAA (51) | AAC (52) | |

Results: The minor alleles of CD44 rs8193 C/T (minor alleles: C/T or T/T), CD166 rs1157 G/A (minor alleles: G/A or A/A) and LGR5 rs17109926 T/C (minor alleles: T/C or C/C) showed significantly longer median TTR (5.7 vs 9.4 yrs, HR 0.66, p=0.024; 5.7 vs 11.3 yrs, HR 0.56, p=0.024; 6.6 vs 10.7 yrs, HR 0.33, p=0.023) in univariate analysis as compared to the major alleles (i.e., C/C for CD44 rs8193 C/T, G/G for CD166 rs1157 G/A, or T/T for LGR5 rs17109926 T/C). After Cox proportional hazards model adjustment for stage and type of adjuvant chemotherapy and stratification by race these results remained significant (HR 0.71, p=0.034; HR 0.55, p=0.027; HR 0.34, p=0.035). No significant association was found between TTR and the remaining tested gene variants.

This example shows that polymorphisms in certain cancer stem cell genes can serve as molecular markers for cancer prognosis, indicating that the analysis of genetic variants of these cancer stem cell genes may help to identify patient subgroups at high risk for tumor recurrence.

### Example 2

The research reported in Example 1 was extended. As noted prevsioudly, a total of 234 patients with stage III and high-risk stage II CC were included in this study. All patients were treated with 5-FU-based adjuvant chemotherapy at the Norris Comprehensive Cancer Center/University of Southern California (NCCC/USC) or the Los Angeles County/USC-Medical Center (LAC/USCMC) from 1987 to 2007. Patient data were collected retrospectively through chart review. Whole blood was collected at the time of diagnosis and stored at −80 degree Celsius. Blood samples from 216 patients were available for current genetic analyses. The study was approved by the Institutional Review Boards at USC and all study participants signed informed consent for the analysis of molecular correlates.

Candidate polymorphisms. Common and putatively functional polymorphisms within genes that have been previously associated with colon CSC were selected using public literature resources and databases including: NCBI-PubMed, dbSNP, Ensembl, GeneCards and the Pharmacogenomics-Knowledge-Base. Stringent and pre-defined selection criteria used were: (a) minor allele frequency ≧10% in Caucasians; (b) polymorphism that could alter the function of the gene in a biologically relevant manner (either published data or predicted function using Functional-Single-Nucleotide-Polymorphism (F-SNP) database, see web address: compbio.cs.queensu.ca/F-SNP (14, 15); (c) published clinical associations (e.g. cancer risk/outcome or chemoresistance). As it was not possible to select all polymorphisms matching these criteria, this study focused on the most promising (Table 2).

Genotyping. Genomic DNA was extracted from peripheral blood using the QIAmp-kit (Qiagen). The majority of the samples were tested using PCR-based restriction fragment length polymorphism (PCR-RFLP) analysis. Forward and reverse primers were used for PCR amplification, PCR products were digested by restriction enzymes (New England Biolabs), and alleles were separated on 4% NuSieve ethidium bromide stained agarose gel. If no matching restriction enzyme could be found, samples were analyzed by direct DNA-sequencing. For quality control purposes, a total of 5% PCR-RFLP analyzed samples were re-analyzed by direct DNA-sequencing. The investigator analyzing the germline polymorphisms was blinded to the clinical dataset.

Statistical analysis. The endpoint of the study was TTR. TTR was calculated from the date of diagnosis of CC to the date of the first observation of tumor recurrence. TTR was censored at the time of death or at the last follow-up if the patient remained tumor recurrence- free at that time. With 216 patients, there was an 80% power to detect a minimum hazard ratio (HR) of 1.8 across the range of minor allele frequencies (0.2-0.5) for TTR using a dominant model. For the recessive model, the minimum HR was 3.1 when the allele frequency was 0.2 and approaches 1.8 when the allele frequency was 0.5. Allelic distribution of the polymorphisms by ethnicity was tested for deviation from Hardy-Weinberg equilibrium using χ²-test. The distribution of polymorphisms across baseline demographic, clinical and pathological characteristics was examined using Fisher's exact test. The true mode of inheritance of all polymorphisms tested is not established yet and Applicant assumed a codominant, additive, dominant or recessive genetic model where appropriate. The association of polymorphisms with TTR was analyzed using Kaplan-Meier curves and log-rank test. In the multivariate Cox-regression analysis, the model was adjusted for stage and type of adjuvant chemotherapy, and stratified by ethnicity. Interactions between polymorphisms and stage and gender on TTR were tested by comparing likelihood ratio statistics between the baseline and nested Cox proportional hazards models that include the multiplicative product term. P-values for all polymorphisms were adjusted for multiple testing using a modified test of Conneely and Boehnke that was applied for the correlated tests due to linkage disequilibrium and the different modes of inheritance considered. (16) Recursive partitioning (RP), including cross-validation, was used to explore and identify polymorphism profiles associated with TTR using the rPart-function in S- plus. Case-wise deletion for missing polymorphisms was used in univariate and multivariate analyses. In the RP-analysis, all patients with at least one polymorphism result available were included. All analyses were performed using SAS 9.2 (SAS Institute Inc. NC, USA).

### Results

The baseline characteristics of the 234 patients included in this analysis are summarized in Table 3. The median age at time of diagnosis was 59 years (range 22-87), with a median follow-up time of 4.4 years (range 0.4-16.8). Ninety (38.5%) patients showed tumor recurrence, with a stage III and high-risk stage II dependent probability of 3-year recurrence of 0.45±0.047 and 0.21±0.043, respectively; the median TTR was 5.2 years (95% CI: 2.6-11.1) and 10.7 years (95% CI: 5.9-16.8), respectively. Median OS has not been reached yet. The genotyping quality control by direct DNA-sequencing provided a genotype concordance of >99%. Genotyping was successful in at least 90% of cases for each polymorphism analyzed, with the exception of CD44 rs8193 (88.4%). In failed cases, genotyping was not successful because of limited quantity and/or quality of extracted genomic DNA. The allelic frequencies for all polymorphisms were within the probability limits of Hardy-Weinberg equilibrium, with the exception of EpCAM rs17036526 (data not shown).

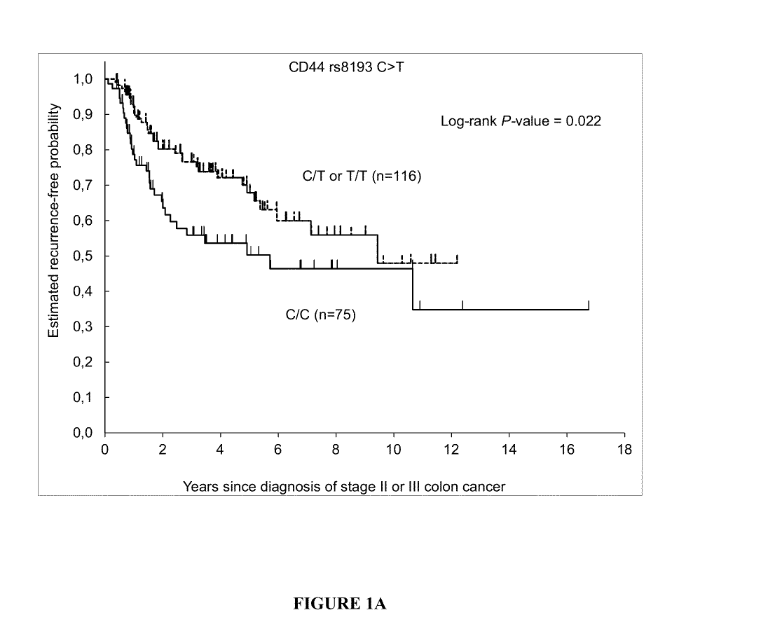

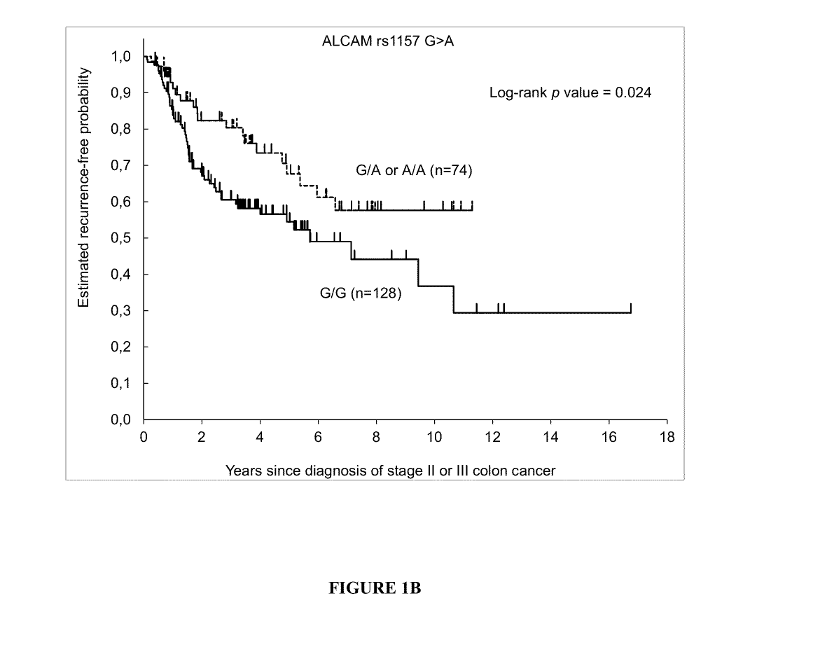

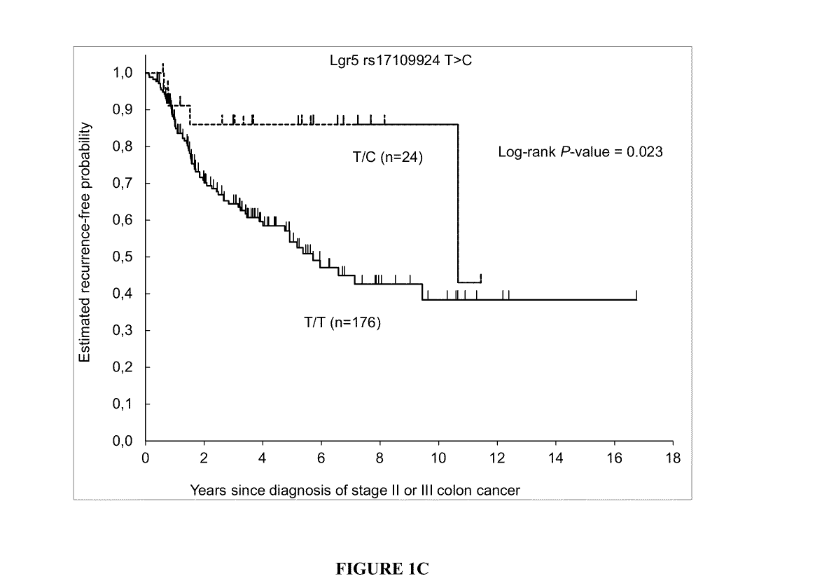

There were no significant associations between the polymorphisms and baseline demographic, clinical or pathological characteristics (data not shown). In the univariate analysis, the minor alleles of CD44 rs8193 C>T, ALCAM rs1157 G>A and LGR5 rs17109924 T>C were significantly associated with an increased TTR. Patients carrying at least one T allele in CD44 rs8193 showed a median TTR of 9.4 years. In contrast, patients with homozygous C/C had a median TTR of 5.4 years (HR, 0.51; 95% CI, 0.35-0.93; P=0.022). Patients harboring the minor allele of ALCAM rs1157 showed a median TTR of 11.3 years compared to 5.7 years for patients harboring the homozygous G/G (HR, 0.56; 95% CI, 0.33-0.94; P=0.024). Patients carrying one C allele in LGR5 rs17109924 had a median TTR of 10.7 years compared to 5.7 years for those patients carrying the homozygous T/T (HR, 0.33; 95% CI, 0.12-0.90; P=0.023; FIG. 1). The other tested gene variants did not demonstrate any statistically significant associations with TTR in the univariate analyses.

In the multivariate analysis stratified by ethnicity, the minor alleles of CD44 rs8193 C>T, ALCAM rs1157 G>A and LGR5 rs17109924 T>C remained significantly associated with increased TTR (Table 4). There was no significant interaction between the polymorphisms and tumor stage or gender on TTR (P-values for interactions >0.05). In multiple testing that including all polymorphisms analyzed, none of them remained significantly associated with TTR (adjusted-P for CD44 rs8193=0.142; adjusted-P for ALCAM rs1157=0.199; adjusted-P for LGR5 rs17109924=0.394).

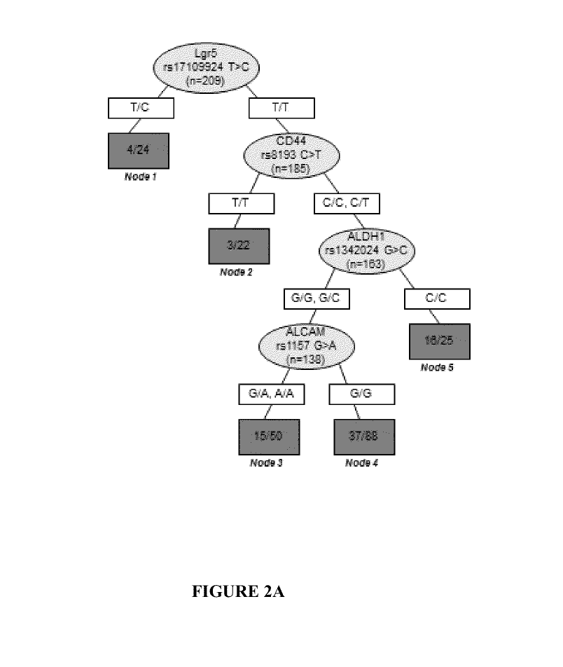

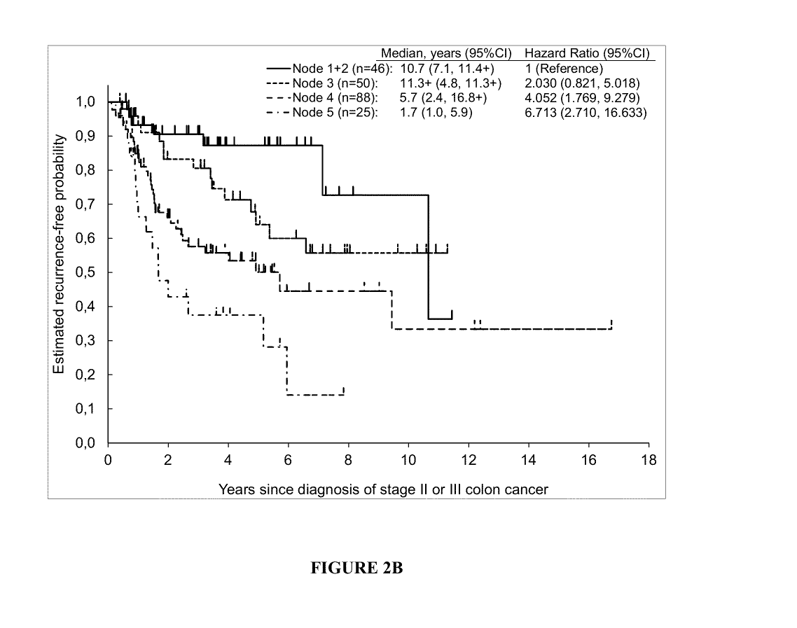

When RP was utilized to construct a decision-tree as a predictive model to classify patients based on the gene variants, high- and low-risk patient subgroups were identified. In the resultant tree, the most important factor that determined the TTR in these patients was LGR5 rs17109924. Patients carrying the combination of LGR5 rs17109924 wild-type and at least one CD44 rs8193 wild-type allele and the mutant variant of ALDH1A1 rs1342024 demonstrated a TTR of 1.7 years (Node 5) compared to 10.7 years in patients with the minor allele of LGR5 rs17109924 (Node 1) or the combination of LGR5 rs17109924 wild-type and CD44 rs8193 mutant variant (Node 2; HR, 6.71, 95% CI, 2.71-16.63, P<0.001; FIG. 2).

To evaluate if the high-risk patients identified from Applicant's gene variant profile benefit from combination chemotherapy (n=47) compared to 5-FU alone (n=66), the cases from node 4 and 5 of the decision tree were combined for further analysis. No significant difference in TTR regarding the treatment regimen were identified in this high-risk subgroup (P>0.05).

### Discussion

In the present study, Applicant investigated germline polymorphisms in a comprehensive panel of genes that have been previously associated with colon CSC to predict tumor recurrence in patients with stage III and high-risk stage II CC. The results indicate that common CSC gene variants in CD44, ALCAM, LGR5 and ALDH1A1 may be valuable to separate high-risk from low-risk CC patients.

The detailed molecular mechanisms involved in how the CD44 rs8193, ALCAM rs1157, LGR5 rs17109924 and ALDH1A1 rs1342024 polymorphisms exert effects on CC are unclear. Non-synonymous polymorphisms lead to amino acid changes and thus may affect the protein function. (Ng et al. (2006) Annu Rev. Genomics Hum. Genet. 7:61-80) 3′UTRs have been implicated in the modulation of gene regulation at the transcriptional level and function as transcriptional regulators mainly through control of mRNA stability and/or translational efficiency, and therefore play an important role in the overall fate of the mRNA. Further, germline polymorphisms in the 3′UTRs have been shown to have functional effects on overall gene expression. (Mandola et al. (2004) Pharmacogenetics 14:319-327) Applicant used the F- SNP database to predict the functional effects of the analyzed polymorphisms. F-SNP gathers computationally predicted functional information about polymorphisms, particularly aiming to facilitate identification of disease-related polymorphisms in association studies. (Lee et al. (2008) Nucleic Acids Res. 36:D820-824; Lee et al. (2009) Bioinformatics 25:1048-1055) When used for this study set of polymorphisms, F-SNP predicted changes in transcriptional regulation for the 3′UTR located CD44 rs8193 and ALCAM rs1157, and changes in splicing regulation and protein coding for the non-synonymous LGR5 rs17109924, thus supporting the translational effects seen in this study. No prediction could be provided for the upstream located ALDH1A1 rs1342024 by the software.

CD44-signaling is crucial in cancer cell proliferation, motility and migration. As a Wnt-target gene, CD44 promotes cell proliferation via the phosphatidylinositol 3-kinase (PI3K)/Akt pathway. CD44 positive CC cells have been reported to possess the capacity for self-renewal, longevity and multipotency. (19) ALCAM belongs to the immunoglobulin superfamily of cell adhesion molecules involved in cell-cell interactions. ALCAM may regulate through cytoskeletal anchoring and the integrity of the extracellular immunoglobulin-like domains complex cellular properties in regard to cell adhesion, migration and growth. (Levin (2010) Gastroenterology 139:2072-2082) Isolation of ALCAM/CD44 double-positive cells from human CC cells can recapitulate tumorigenesis when xenografted at low numbers into immune- deficient mice which represents a hallmark of CSCs. (Dalerba et al. (2007) Proc. Natl. Acad. Sci. U.S.A. 104:10158-10163) Despite the potentially high clinical relevance of these CSC markers, little is known about their prognostic significance in CC and contradictory findings have been reported. In a recent study based on 110 CRC patients, membranous expression of CD44 and ALCAM did not predict survival in single-marker analyses, but gained significance when combined. (Horst et al. (2009) Cancer Invest. 27:844-850) In contrast, Weichert et al. found a correlation between membranous ALCAM expression and decreased survival in 111 CRC patients. (Weichert et al. (2004) J. Clin. Pathol. 57:1160-1164) Interestingly, loss, rather than overexpression, of membranous CD44 and ALCAM was correlated to outcome in an analysis including 101 CRC patients. The authors suggested that their results are in large part dependent on the cell adhesion function of CD44 and ALCAM with loss of cell adhesion representing a fundamental step underlying the initiation of the metastatic process. (Lugli et al. (2010) Br. J. Cancer 103:382-390)

These conflicting results raised the question whether germline genetic variants putatively changing the gene's function rather than membranous evaluation of these proteins may predict CC patient's outcome. Applicant recently showed that the minor allele of CD44 rs187116 predicts decreased TTR and OS in patients with localized gastric adenocarcinoma. (Winder et al. (2010) Int. J. Cancer) More recently, Zhou et al. analyzed two polymorphisms in ALCAM investigating 1033 breast cancer patients and 1116 controls and found that individuals harboring the ALCAM rs6437585 C/T or T/T genotypes have an odds ratio (OR) of 1.38 (95% CI, 1.11-1.72) for developing breast cancer, compared to the C/C genotype. Additional experiments showed that the T allele was associated with a higher transcriptional activity of the ALCAM gene. (Zhou et al. (2010) Breast Cancer Res. Treat.) Both, polymorphisms CD44 rs187116 and ALCAM rs6437585, did not show any clinical associations in this study. However, these SNPs have been analyzed in other tumor entities and/or setting (risk assessment) and therefore might not exert their effects in CC and tumor recurrence assessment. Since CD44 rs8193 and ALCAM rs1157 may change transcriptional regulation as predicted by F-SNP, the findings in the present study that these gene variants affect TTR in CC are biologically plausible.

Interestingly, the tree analysis provided LGR5 rs17109924 as the first split indicating the most important factor determining TTR in this patient cohort. LGR5 is a member of the G- protein-coupled receptor (GPCR) family comprising proteins with seven transmembrane domains. GPCRs function as receptors for various classes of ligands, including peptide hormones and chemokines; however, the ligand for and function of LGR5-related signaling remains unclear. LGR5, a Wnt-target gene, has been reported to be a marker for colon CSC, thus playing a putative role in the biological function of stem cells. In a recent study, high membranous LGR5 expression was shown to predict lower DFS in CRC patients. (25-27) LGR5 rs17109924 represents a non-synonymous SNP and was predicted to affect splicing regulation and protein coding by F-SNP. In addition, LGR5 rs17109924 predicted TTR in both the univariate and multivariate analysis and was incorporated in the tree analysis, strongly indicating that this SNP has functional significance.

A combination of gene variants in the tree analysis defined a high-risk subgroup with significantly lower TTR by incorporating ALDH1A1 rs1342024 when compared to single marker analysis. ALDH1 is a detoxifying enzyme that oxidizes intracellular aldehydes. This detoxification capacity may protect stem cells against oxidative insult. (28, 29) In a recent study, membranous ALDH1 expression did not predict survival in CRC patients. (23) Although the mechanism of ALDH1A1 rs1342024 remains unclear, our data suggests that a multigenic approach, which assesses the combined effects of gene variants, may detect synergistic interactions between individual SNPs thus enhancing the predictive power of the model.

This study further utilized multiple testing due to the large number of independent genetic variants evaluated. Application of a modified test of Conneely and Boehnke resulted in a non-significant adjusted P-value for CD44 rs8193, ALCAM rs1157 and LGR5 rs17109924. Therefore, these data warrant further validation in a larger cohort. Nevertheless, the biological plausibility and Applicant's translational findings hold promise for further investigations in independent study populations. In a sub-analysis combining high-risk patients based on Applicant's gene variant profile, no benefit for the addition of oxaliplatin or irinotecan to 5-FU- based chemotherapy could be demonstrated. Since all patients included in this study represent stage III and high-risk stage II CC treated with adjuvant therapy, it was not possible to correlate the genotypes with clinical outcome in an untreated control group. As a consequence, it could not be determined whether the high-risk patients, based on the gene variant profile, did not benefit from combination chemotherapy or from chemotherapy at all.

This study provides the first evidence that germline polymorphisms in CSC genes predict early tumor recurrence in patients with CC. This may help to select subgroups of patients who may benefit from more aggressive treatment strategies or newly developed stem cell targeting drugs. Future biomarker-embedded translational trials are warranted to validate these findings.

In conclusion, to the best of Applicants' knowledge, this is the first study identifying common germline variants in colon CSC genes as independent prognostic markers for stage III and high-risk stage II colon cancer patients.

TABLE 2
Analyzed CSC gene polymorphisms

| | | Base | | |
|---|---|---|---|---|
| Gene | rs-number | exchange | Region | Genotyping |
| CD44 | rs8193 | C > T | 3UTR | RE (BsrDI) |
| | rs187116 | A > G | Intron | RE (MspI) |
| | rs4755392 | T > A | 3UTR | DS |
| | rs7116432 | A > G | 3UTR | RE (NlaIII) |
| Prominin-1 | rs3130 | A > G | 3UTR | RE (EcorRI) |
| | rs2240688 | A > C | 3UTR | DS |
| | rs2286455 | C > T | Splice Site | RE (MboI) |
| DPP4 | rs2300757 | G > C | Intron | RE (TfiI) |
| | rs1014444 | A > G | Intron | RE (AluI) |
| | rs2268894 | A > G | Intron | DS |
| EpCAM | rs17036526 | G > C | Splice site | RE (DdeI) |
| | rs1126497 | C > T | Non- | RE (NiaIII) |
| | | | synonymous | |
| | | | coding | |
| | rs1421 | T > C | 3UTR | DS |
| ALCAM | rs6437585 | C > T | 5UTR | RE (Cac8I) |
| | rs1044240 | A > G | Non- | DS |
| | | | synonymous | |
| | | | coding | |
| | rs1044243 | G > A | Non- | DS |
| | | | synonymous | |
| | | | coding | |
| | rs1157 | G > A | 3UTR | RE (MspI) |
| MSI-1 | rs2522137 | A > C | 3UTR | DS |
| ITGB1 | rs2153875 | T > G | Splice site | RE (BfaI) |
| CD24 | rs8734 | C > T | Non- | RE (AciI) |
| | | | synonymous | |
| | | | coding | |
| | rs3838646 | —/CA | 3UTR | RE (BsrI) |
| LGR5 | rs17109924 | T > C | Non- | DS |
| | | | synonymous | |
| | | | coding | |
| | rs17109926 | G > A | 3UTR | DS |
| ALDH1A1 | rs13959 | G > A | Synonymous | RE (Hypch4III) |
| | | | coding | |
| | rs1342024 | G > C | Upstream | DS |

Abbreviations: DS, direct DNA sequencing; RE, restriction enzyme; UTR, untranslated region; DPP4, dipeptidyl peptidase-4; EpCAM, epithelial cell adhesion molecule; ALCAM, activated leukocyte cell adhesion molecule; MSI-1, musashi homolog-1; ITGB1, integrin beta-1; LGR5, leucine-rich repeat containing G protein-coupled receptor-5; ALDH1A1, aldehyde dehydrogenase-1 family member A1

TABLE 3
Baseline patient characteristics

| | n | % |
|---|---|---|
| Sex | | |
| Female | 107 | 45.72 |
| Male | 127 | 54.28 |
| Ethnicity | | |
| Asian | 34 | 14.53 |
| African American | 15 | 6.41 |
| Caucasian | 123 | 52.56 |
| Hispanic | 62 | 26.5 |
| T | | |
| T1 | 2 | 0.85 |
| T2 | 14 | 5.98 |
| T3 | 187 | 79.91 |
| T4 | 27 | 11.54 |
| Tx | 4 | 1.72 |
| Grade | | |
| Well | 11 | 2.18 |
| Moderate | 151 | 64.53 |
| Poor/undifferentiated | 54 | 23.08 |
| Missing | 18 | 10.21 |
| N | | |
| Negative | 105 | 44.87 |
| N1 | 72 | 30.77 |
| N2 | 57 | 24.36 |
| Stage | | |
| High-risk II | 105 | 44.87 |
| III | 129 | 55.13 |
| N of resected lymph nodes | | |
| ≦12 | 70 | 29.91 |
| >12 | 145 | 61.97 |
| Missing | 19 | 8.12 |
| Tumor side | | |
| Left | 110 | 47.01 |
| Right | 115 | 49.15 |
| Left and right | 4 | 1.71 |
| Missing | 5 | 2.13 |
| Adjuvant treatment | | |
| 5-FU | 151 | 64.53 |
| 5-FU/LV/Oxaliplatin | 60 | 25.64 |
| 5-FU/LV/Irinotecan | 23 | 9.83 |

TABLE 4
Univariate and multivariate analysis of polymorphisms predicting TTR

| | | Time to tumor recurrence | | | | |
|---|---|---|---|---|---|---|
| | | Univariate analysis | | | | |
| | | Median | | | Multivariate analysis | |
| | | TTR, yrs | HR (95% | P | | P |
| | N | (95% CI) | CI) | value | HR (95% CI) | value |
| CD44 rs8193 | | | | | | |
| C/C | 75 | 5.4 (2.1-16.8+) | 1 (Reference) | | 1 (Reference) | |
| C/T, T/T | 116 | 9.4 (5.9-12.2) | 0.51 (0.35-0.93) | 0.022 | 0.60 (0.36-0.99) | 0.047 |
| ALCAM | | | | | | |
| rs1157 | | | | | | |
| G/G | 128 | 5.7 (3.2-10.7) | 1 (Reference) | | 1 (Reference) | |
| G/A, A/A | 74 | 11.3 (5.9-11.3+) | 0.56 (0.33-0.94) | 0.024 | 0.55 (0.32-0.93) | 0.027 |
| LGR5 | | | | | | |
| rs17109924 | | | | | | |
| T/T | 176 | 5.7 (4.0-16.8) | 1 (Reference) | | 1 (Reference) | |
| T/C | 24 | 10.7 (10.7-11.4+) | 0.33 (0.12-0.90) | 0.023 | 0.33 (0.12-0.93) | 0.035 |

Abbreviations:
TTR, time to tumor recurrence;
HR, hazard ratio;
ALCAM, activated leukocyte cell adhesion molecule;
LGR5, leucine-rich repeat containing G protein-coupled receptor 5.

The disclosure illustratively described herein may suitably be practiced in the absence of any element or elements, limitation or limitations, not specifically disclosed herein. Thus, for example, the terms “comprising”, “including,” containing“, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosure embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Cunningham D, Atkin W, Lenz H J, et al: Colorectal cancer. Lancet 375:1030-47, 2010
2. Tejpar S, Bertagnolli M, Bosman F, et al: Prognostic and predictive biomarkers in resected colon cancer: current status and future perspectives for integrating genomics into biomarker discovery. Oncologist 15:390-404, 2010
3. Zeki S S, Graham T A, Wright N A: Stem cells and their implications for colorectal cancer. Nat Rev Gastroenterol Hepatol 8:90-100, 2011
4. Todaro M, Francipane M G, Medema J P, et al: Colon cancer stem cells: promise of targeted therapy. Gastroenterology 138:2151-62, 2010
5. Dalerba P, Dylla S J, Park I K, et al: Phenotypic characterization of human colorectal cancer stem cells. Proc Natl Acad Sci USA 104:10158-63, 2007
6. Sanders M A, Majumdar A P: Colon cancer stem cells: implications in carcinogenesis. Front Biosci 16:1651-62, 2010
7. Vermeulen L, Todaro M, de Sousa Mello F, et al: Single-cell cloning of colon cancer stem cells reveals a multi-lineage differentiation capacity. Proc Natl Acad Sci USA 105:13427-32, 2008
8. Saif M W, Chu E: Biology of colorectal cancer. Cancer J 16:196-201, 2010
9. Coate L, Cuffe S, Horgan A, et al: Germline genetic variation, cancer outcome, and pharmacogenetics. J Clin Oncol 28:4029-37, 2010
10. Winder T, Ning Y, Yang D, et al: Germline polymorphisms in genes involved in the CD44 signaling pathway are associated with clinical outcome in localized gastric adenocarcinoma (GA). Int J Cancer, 2010
11. McShane L M, Altman D G, Sauerbrei W, et al: REporting recommendations for tumor MARKer prognostic studies (REMARK). Breast Cancer Res Treat 100:229-35, 2006
12. Alonzo T A: Standards for reporting prognostic tumor marker studies. J Clin Oncol 23:9053-4, 2005
13. McShane L M, Altman D G, Sauerbrei W, et al: Reporting recommendations for tumor marker prognostic studies. J Clin Oncol 23:9067-72, 2005
14. Lee P H, Shatkay H: F-SNP: computationally predicted functional SNPs for disease association studies. Nucleic Acids Res 36:D820-4, 2008
15. Lee P H, Shatkay H: An integrative scoring system for ranking SNPs by their potential deleterious effects. Bioinformatics 25:1048-55, 2009
16. Conneely K N, Boehnke M: So Many Correlated Tests, So Little Time! Rapid Adjustment of P Values for Multiple Correlated Tests. Am J Hum Genet 81, 2007
17. Ng P C, Henikoff S: Predicting the effects of amino acid substitutions on protein function. Annu Rev Genomics Hum Genet 7:61-80, 2006
18. Mandola M V, Stoehlmacher J, Zhang W, et al: A 6 by polymorphism in the thymidylate synthase gene causes message instability and is associated with decreased intratumoral TS mRNA levels. Pharmacogenetics 14:319-27, 2004
19. Du L, Wang H, He L, et al: CD44 is of functional importance for colorectal cancer stem cells. Clin Cancer Res 14:6751-60, 2008
20. Levin T G, Powell A E, Davies P S, et al: Characterization of the intestinal cancer stem cell marker CD166 in the human and mouse gastrointestinal tract. Gastroenterology 139:2072-2082 e5, 2010
21. Horst D, Kriegl L, Engel J, et al: Prognostic significance of the cancer stem cell markers CD133, CD44, and CD166 in colorectal cancer. Cancer Invest 27:844-50, 2009
22. Weichert W, Knosel T, Bellach J, et al: ALCAM/CD166 is overexpressed in colorectal carcinoma and correlates with shortened patient survival. J Clin Pathol 57:1160-4, 2004
23. Lugli A, Iezzi G, Hostettler I, et al: Prognostic impact of the expression of putative cancer stem cell markers CD133, CD166, CD44s, EpCAM, and ALDH1 in colorectal cancer. Br J Cancer 103:382-90, 2010
24. Zhou P, Du L F, Lv GQ, et al: Functional polymorphisms in CD166/ALCAM gene associated with increased risk for breast cancer in a Chinese population. Breast Cancer Res Treat, 2010
25. Barker N, Clevers H: Leucine-rich repeat-containing G-protein-coupled receptors as markers of adult stem cells. Gastroenterology 138:1681-96, 2010
26. Uchida H, Yamazaki K, Fukuma M, et al: Overexpression of leucine-rich repeat- containing G protein-coupled receptor 5 in colorectal cancer. Cancer Sci 101:1731-7, 2010
27. Takahashi H, Ishii H, Nishida N, et al: Significance of LGR5(+ve) Cancer Stem Cells in the Colon and Rectum. Ann Surg Oncol 18:1166-74, 2010
28. Huang E H, Hynes M J, Zhang T, et al: Aldehyde dehydrogenase 1 is a marker for normal and malignant human colonic stem cells (SC) and tracks SC overpopulation during colon tumorigenesis. Cancer Res 69:3382-9, 2009
29. Deng S, Yang X, Lassus H, et al: Distinct expression levels and patterns of stem cell marker, aldehyde dehydrogenase isoform 1 (ALDH1), in human epithelial cancers. PLoS One 5:e10277, 2010

1. A method for aiding in the determination of or determining whether a cancer patient is likely to experience a longer or shorter time to tumor recurrence, comprising screening a tissuc or cell sample isolated from the patient for at least one polymorphism of CD44 rs8193 C/T (SEQ ID NO: 53), ALCAM rs1157 G/A (SEQ ID NO: 54) or LGR5 rs rs17109924 T/C (SEP ID NO: 55), wherein the presence of one or more genotypes of:
(a) (T/T or C/T) for CD44 rs8193 C/T (SEQ ID NO: 53);
(b) (A/A or G/A) for ALCAM rs1157 G/A (SEQ ID NO: 54); or
(c) (C/C or T/C) for LGR5 rs17109924 T/C (SEQ ID NO: 55);
determines that the patient is likely to experience a longer time to tumor recurrence, or the presence of none of genotypes (a)-(c) determines that the patient is likely to experience a shorter time to tumor recurrence.

2. The method of claim 1, wherein the presence of one or more genotypes of:
(a) (T/T or C/T) for CD44 rs8193 C/T (SEQ ID NO: 53);
(b) (A/A or G/4\ for ALCAM rs1157 G/A (SEQ ID NO: 54);
(c) (C/C or T/C) for LGR5 rs17109924 T/C (SEQ ID NO: 55);
determines that the patient is likely to experience a longer time to tumor recurrence.

3. The method of claim 1 or 2, wherein a patient likely to experience a longer time to tumor recurrence is as compared to a patient suffering from a same cancer and having none of genotypes (a)-(c).

4. The method of claim 1, wherein the presence of none of genotypes (a)-(c) determines that the patient is likely to experience a shorter time to tumor recurrence.

5. The method of claim 1, wherein a patient likely to experience a shorter time to tumor recurrence is as compared to a patient suffering from a same cancer and having one or more genotypes of (a)-(c).

6. The method of claim 1, wherein the patient suffers from one or more cancer selected from lung cancer, non-small cell lung cancer, breast cancer, head and neck cancer, ovarian cancer, colon cancer, Stage II or Stage III colon cancer, localized gastric cancer, gastric adenocarcinoma, rectal cancer, colorectal cancer, esophageal cancer, gastric cancer, liver cancer, bone cancer, spleen cancer, pancreatic cancer, or gallbladder cancer.

7. The method of claim 1, wherein the patient suffers from one or more colorectal cancer.

8. The method of claim 7, wherein the gastrointestinal cancer is colon cancer.

9. The method of claim 1, wherein the patient is a Stage II or III colon cancer patient.

10. The method of claim 1, wherein the sample comprises at least one of a tumor cell or tumor tissue, a normal cell or normal tissue, a normal cell or normal tissue adjacent to a tumor, a normal cell or normal tissue corresponding to the tumor tissue type, a blood cell, a peripheral blood lymphocyte, or combinations thereof.

11. The method of claim 1, wherein the sample is at least one of a cell or tissue sample recently isolated from the patient, a fixed tissue, a frozen tissue, a biopsy tissue, a resection tissue, a microdissected tissue, or combinations thereof.

12. The method of claim 1, wherein the screening is by a method comprising polymerase chain reaction analysis (PCR), sequencing analysis, restriction enzyme analysis, mismatch cleavage analysis, single strand conformation polymorphism analysis, denaturing gradient gel electrophoresis, selective oligonucleotide hybridization, selective PCR amplification, selective primer extension, oligonucleotide ligation assay, exonuclease-resistant nucleotide analysis, Genetic Bit Analysis, primer-guided nucleotide incorporation analysis PCR, PCR-restriction fragment length polymorphism (PCR- RFLP), direct DNA sequencing, whole genome sequencing, and/or microarray.

13. The method of claim 1, wherein the patient is an animal patient.

14. The method of claim 13, wherein the patient is of the group of a mammalian, a human, a simian, a murine, a bovine, an equine, a porcine, a feline, a canine, or an ovine patient.

15. The method of claim 14, wherein the patient is a human patient.

16. A kit for use in aiding in the determination of or determining whether a cancer patient is likely to experience a longer or shorter time to tumor recurrence, comprising suitable primers or probes or a microarray for screening a tissue or cell sample isolated from the patient for at least one polymorphism of CD44 rs8193 C/T (SEQ ID NO: 53), ALCAM rs1157 G/A (SEQ ID NO: 54) or LGR5 rs17109924 T/C (SEQ ID NO: 55), and instructions for use therein.

17. A prognostic panel of primer or probes or a microarray comprising nucleic acids that identify the genotype in a patient sample for determining at least two polymorphisms of CD44 rs8193 C/T (SEQ ID NO: 53), ALCAM rs1157 G/A (SEQ ID NO: 54) or LGR5 rs17109924 T/C (SEQ ID NO: 55).
